(19) United States
(12) Patent Application Publication
(10) Pub. No.: US 2010/0311184 A1
(43) Pub. Date: Dec. 9, 2010

(54) AZO HETEROCYCLIC DYES AND THEIR BIOLOGICAL CONJUGATES

(75) Inventors: Zhenjun DIWU, Sunnyvale, CA (US); Jingfang LIAO, Foster City, CA (US)

Correspondence Address: KOLISCH HARTWELL, P.C., 200 PACIFIC BUILDING, 520 SW YAMHILL STREET, PORTLAND, OR, 97204, US

(73) Assignee: AAT BIOQUEST, INC., Sunnyvale, CA (US)

(21) Appl. No.: 12/791,616

(22) Filed: Jun. 1, 2010

Related U.S. Application Data
(60) Provisional application No. 61/217,759, filed on Jun. 4, 2009.

(51) Int. Cl. G01N 21/76; C09B 29/00; C07K 7/06

(52) U.S. Cl. 436/172; 534/795; 534/774; 534/727; 530/328

(57) ABSTRACT

Azo heterocyclic compounds that are substantially non-fluorescent or only weakly fluorescent useful as energy acceptors. The chemically reactive compounds possess utility for labeling a wide variety of substances, including biomolecules with the resulted conjugates being highly useful for a variety of energy-transfer assays and applications. The biological conjugates of the disclosure exhibit little or no observable fluorescence and their fluorescence is significantly increased upon a biological stimulation.

### CROSS-REFERENCE TO RELATED APPLICATIONS

This application claims the benefit under 35 U.S.C. §119(e) of U.S. Provisional Application Ser. No. 61/217,759 entitled AZO HETEROCYCLIC DYES AND THEIR BIOLOGICAL CONJUGATES filed Jun. 4, 2009, which is hereby incorporated by reference.

### BACKGROUND

Fluorescence Resonance Energy Transfer (FRET) is a process whereby a first fluorescent dye (the “donor” dye) is excited, typically by illumination, and transfers its absorbed energy to a second dye (the “acceptor” dye) having a longer wavelength and therefore lower energy emission. Where the second dye is fluorescent, this energy transfer may result in fluorescence at the emission wavelength of the second dye. However, where the second dye is nonfluorescent, the absorbed energy does not result in fluorescence emission, and the fluorescence of the initial donor dye is said to be “quenched”. Energy transfer can also be utilized to quench the emission of fluorescent donors, including phosphorescent and chemifluorescent donors. When a fluorescent emission is restored by preventing energy transfer, the fluorescence is said to be “dequenched” or “unquenched”.

Techniques employing FRET have been utilized to study DNA hybridization and amplification, the dynamics of protein folding, proteolytic degradation, and interactions between other biomolecules (Methods in Enzymology, Vol. 278). A common donor-acceptor dye pair utilized for these applications is dabcyl (the quenching dye) and EDANS (the fluorophore). Selected examples of biological applications of FRET can be found in the following references, among others:
(1) Holskin, B. P.; Bukhtiyarova, M.; Dunn, B. M.; Baur, P.; Dechastonay, J.; Pennington, M. W. Anal Biochem 1995, 227, 148-155.
(2) Beekman, B.; Drijfhout, J. W.; Bloemhoff, W.; Ronday, H. K.; Tak, P. P.; to Koppele, J. M. FEBS Lett 1996, 390, 221-225.
(3) Pennington, M. W.; Thomberry, N. A. Peptide Research 1994, 7, 72-76.
(4) Wang, Q. M.; Johnson, R. B.; Cohen, J. D.; Voy, G. T.; Richardson, J. M.; Jungheim, L. N. Antivir Chem Chemother 1997, 8, 303-310.
(5) Gulnik, S. V.; Suvorov, L. I.; Majer, P.; Collins, J.; Kane, B. P.; Johnson, D. G.; Erickson, J. W. FEBS Lett 1997, 413, 379-384.
(6) Beekman, B.; van El, B.; Drijfhout, J. W.; Ronday, H. K.; TeKoppele, J. M. FEBS Lett 1997, 418, 305-309.
(7) Beebe, K. D.; Pei, D. Anal Biochem 1998, 263, 51-56.

Unfortunately, the low wavelength excitation used for the dabcyl-EDANS dye pair is not optimal due to the autofluorescence exhibited by most cellular systems, and ultraviolet light can also cause DNA cross-linking in some systems. Many drugs, potential drugs, and biologically active proteins also have very strong absorptions in the low wavelength region. In addition dabcyl and EDANS have low extinction coefficients, resulting in assays that are comparatively insensitive.

### BRIEF DESCRIPTION OF THE DRAWINGS

FIG. 1 is a reaction scheme showing an exemplary synthesis of selected azo-heterocyclic phosphoramidite dyes

FIG. 2 is a reaction scheme showing an exemplary synthesis of selected azo-heterocyclic dyes conjugated to a CPG polymer support

FIG. 3 is a plot showing how HIV protease-mediated cleavage of substrate peptides can be monitored via fluorescence resonance energy transfer (FRET) using azo-heterocyclic dyes, as described in Example 28.

FIG. 4 is a plot showing how matrix metalloproteinase (MMP) mediated cleavage of substrate enzyme substrates can be monitored by fluorescence using azo-heterocyclic dyes, as described in Example 27.

FIG. 5 is a plot showing carbachol dose response in HEK-293 cells measured with Screen Quest™ Fluo-8 NW Assay kit in combination with Compound 42, as described in Example 29.

### DETAILED DESCRIPTION AND DESCRIPTION OF PREFERRED EMBODIMENTS

In order to avoid the difficulties associated with the use of ultraviolet excitation, what is needed is an energy acceptor having an absorption maximum that is aligned with the emission of the fluorophore used. The energy acceptor should quench a large variety of dyes, including dyes that are excited in the ultraviolet as well as at longer wavelengths The compounds of the present disclosure are azo dyes, that is they are compounds that incorporate the chemical group R—N═N—R′, where at least one of R and R′ is a 5-membered heterocyclic moiety. The resulting azo heterocyclic dye may be substituted by a chemically reactive moiety (RM).

These azo heterocyclic compounds have been discovered to quench the fluorescence of a large variety of dyes, including dyes that are excited in the ultraviolet, but also including fluoresceins, rhodamines, and even longer wavelength fluorophores such as Cy5 and allophycocyanin. In addition, the compounds of the disclosure have significantly larger extinction coefficients than the quenching compounds that are typically currently used in energy transfer assays, but are substantially non-fluorescent, or only weakly fluorescent. These compounds therefore represent a new and highly useful class of energy acceptors, including chemically reactive versions, and conjugates prepared therefrom.

In one aspect, the compounds of the disclosure may be described by Formula I:

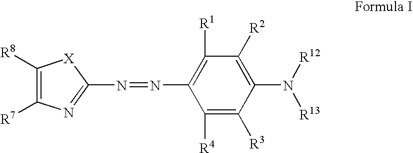

where X is CH₂, CH—R¹⁰, C—R¹⁰R¹¹, O, S, Se, NH or N—R¹⁰.

Dye substituents R¹ to R⁸ are independently hydrogen, alkyl having from 1-20 carbons, alkoxy having from 1-20 carbons, trifluoromethyl, halogen, methylthio, sulfonyl, phosphonyl, boronyl, carbonyl, hydroxy, amino, thiol, aryl or an RM; R¹⁰ to R¹³ are independently alkyl, polyethylene glycol, aryl or an RM; Alternatively, or in addition, the combination of R¹ with R², R³ with R⁴, and/or R⁷ with R⁸ result in one or more saturated rings, unsaturated rings, or aromatic rings. Alternatively, the combination of R⁷ with one or more of R¹-R⁴ or R⁸ with one or more of R¹-R⁴ may result in a more rigid structure that is more stable than the corresponding open structure.

Alternatively or in addition, one or more of R¹ to R⁸ is, or is substituted by a charged moiety. A charged moiety is a functional group that possesses a formal positive or negative electronic charge, for example sulfonate and ammonium substituents. The presence of one or more charged moieties typically increases the water solubility of the azo heterocyclic dye.

As used herein, by “sulfo” is meant sulfonic acid, or salt of sulfonic acid (sulfonate). Similarly, by “carboxy” is meant carboxylic acid or salt of carboxylic acid. “Phosphate”, as used herein, is an ester of phosphoric acid, and includes salts of phosphate. “Phosphonate”, as used herein, means phosphonic acid and includes salts of phosphonate. As used herein, unless otherwise specified, the alkyl portions of substituents such as alkyl, alkoxy, arylalkyl, alylamino, dialkylamino, trialkylammonium, or perfluoroalkyl are optionally saturated, unsaturated, linear or branched, and all alkyl, alkoxy, alkylamino, and dialkylamino substituents are themselves optionally further substituted by carboxy, sulfo, amino, or hydroxy.

For all compounds of Formula I, at least one of R¹ to R⁸ and X is or is substituted by a chemically reactive moiety (RM) or a conjugated substance.

### Counterions

Many embodiments of the compounds of the disclosure possess an overall electronic charge. It is to be understood that when such electronic charges are shown to be present, they are balanced by the presence of one or more appropriate counterions, which may or may not be explicitly identified. In some aspects, the appropriate counterion is a biologically compatible counterion. As used herein, a biologically compatible counterin is not toxic in biological applications, and does not have a substantially deleterious effect on biomolecules. Where the compound of the disclosure is positively charged, the counterion is typically selected from, but not limited to, chloride, bromide, iodide, sulfate, alkanesulfonate, arylsulfonate, phosphate, perchlorate, tetrafluoroborate, tetraarylboride, nitrate, and anions of aromatic or aliphatic carboxylic acids. Where the compound of the disclosure is negatively charged, the counterion is typically selected from, but not limited to, alkali metal ions, alkaline earth metal ions, transition metal ions, ammonium or substituted ammonium ions, or pyridinium ions. Preferably, any necessary counterion is biologically compatible, is not toxic as used, and does not have a substantially deleterious effect on biomolecules. Counterions are readily changed by methods well known in the art, such as ion-exchange chromatography, or via selective precipitation.

The compounds of the disclosure may be represented by chemical formulae that represent one or another particular electronic resonance structure. It should be understood that every aspect of the description of the compounds of the disclosure applies equally to compounds that are related as formal resonance structures, as the electronic charge on the subject dyes are typically delocalized throughout the compound.

### Chemically Reactive Moieties

In one aspect, the compounds of the disclosure include at least one chemically reactive moiety (RM). The RM is chemically reactive functional group selected to cross-react with one or more types of functional groups to form a covalent bond or other linkage, typically creating a dye-conjugate.

The RM is attached to the compound of the disclosure by a covalent linkage L. L may be a single covalent bond, or the covalent linkage L may include multiple intervening atoms forming the covalent linkage. In one aspect, the covalent linkage L includes sufficient intervening atoms to serve as a spacer between the compound of the disclosure and the substance it is conjugated with. Typically, the conjugation reaction between the reactive compound and the substance to be conjugated results in one or more atoms of the reactive group RM becoming incorporated into a new linkage L′ attaching the compound to the conjugated substance.

Those compounds that include an RM are capable of labeling a wide variety of organic or inorganic substances that contain or are modified to contain functional groups with suitable reactivity, resulting in chemical attachment of the conjugated substance. Typically the reactive group is an electrophile or nucleophile that can form a covalent linkage through exposure to the corresponding functional group that is a nucleophile or electrophile, respectively. Alternatively, the reactive group is a photoactivatable group, and becomes chemically reactive only after illumination with light of an appropriate wavelength.

Selected examples of reactive groups and linkages are shown in Table 1, where the reaction of an electrophilic group and a nucleophilic group yields a covalent linkage.

TABLE 1
Examples of RM groups to useful covalent linkages

| Electrophilic Group | Nucleophilic Group | Resulting Conjugate |
|---|---|---|
| activated esters* | amines/anilines | carboxamides |
| acrylamides | thiols | thioethers |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic adds | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

*Activated esters, as understood in the art, generally have the formula —COW, where W is a good leaving group (e.g. succinimidyloxy (—OC₄H₄O₂) sulfosuccinimidyloxy (—OC₄H₃O₂—SO₃H), -1-oxybenzotriazolyl (—OC₆H₄N₃); or an aryloxy group or aryloxy substituted one or more times by electron withdrawing substituents such as nitro, fluoro, chloro, cyano, or trifluoromethyl, or combinations thereof, used to form activated aryl esters; or a carboxylic acid activated by a carbodiimide to form an anhydride or mixed anhydride —OCOAlk or —OCN(Alk₁)NH(Alk₂), where Alk₁ and Alk₂, which may be the same or different, are C₁-C₂₀ alkyl, C₁-C₂₀ perfluoroalkyl, or C₁-C₂₀ alkoxy; or cyclohexyl, 3-dimethylaminopropyl, or N-morpholinoethyl).
**Acyl azides can also rearrange to isocyanates.

The selection of a particular reactive group to attach the compound of the disclosure to the substance to be conjugated typically depends on the functional group present on the substance to be conjugated, and the type or length of covalent linkage desired. The types of functional groups typically present on the organic or inorganic substances include, but are not limited to, amines, amides, thiols, alcohols, phenols, aldehydes, ketones, phosphates, imidazoles, hydrazines, hydroxylamines, disubstituted amines, halides, epoxides, carboxylate esters, sulfonate esters, purines, pyrimidines, carboxylic acids, olefinic bonds, or a combination of these groups. A single type of reactive site may be available on the substance (typical for polysaccharides), or a variety of sites may be present (e.g. amines, thiols, alcohols, phenols), as is typical for proteins. A conjugated substance may be conjugated to more than one dye, which may be the same or different, or to a substance that is additionally modified by a hapten, such as biotin. Although some selectivity can be obtained by careful control of the reaction conditions, selectivity of labeling is best obtained by selection of an appropriate reactive dye.

Typically, the RM is selected to react with an amine, a thiol, an alcohol, an aldehyde or a ketone. Preferably RM is selected to react with an amine or a thiol functional group. In one embodiment, RM is an acrylamide, a reactive amine (including a cadaverine or ethylenediamine), an activated ester of a carboxylic acid (typically a succinimidyl ester of a carboxylic acid), an acyl azide, an acyl nitrile, an aldehyde, an alkyl halide, an anhydride, an aniline, an aryl halide, an azide, an aziridine, a boronate, a carboxylic acid, a diazoalkane, a haloacetamide, a halotriazine, a hydrazine (including hydrazides), an imido ester, an isocyanate, an isothiocyanate, a maleimide, a phosphoramidite, a reactive platinum complex, a sulfonyl halide, or a thiol group. By “reactive platinum complex” is particularly meant chemically reactive platinum complexes such as is described in U.S. Pat. Nos. 5,580,990; 5,714,327; and 5,985,566 (all hereby incorporated by reference).

Where the reactive moiety RM is a photoactivatable group, such as an azide, diazirinyl, azidoaryl, or psoralen derivative among others, the substituted dye becomes chemically reactive only after illumination with light of an appropriate wavelength. Where RM is an activated ester of a carboxylic acid, the reactive dye is particularly useful for preparing dye-conjugates of proteins, nucleotides, oligonucleotides, or haptens. Where RM is a maleimide or haloacetamide the reactive dye is particularly useful for conjugation to thiol-containing substances. Where RM is a hydrazide, the reactive dye is particularly useful for conjugation to periodate-oxidized carbohydrates and glycoproteins, and in addition is an aldehyde-fixable polar tracer for cell microinjection. Preferably, RM is a carboxylic acid, a succinimidyl ester of a carboxylic acid, a haloacetamide, a hydrazine, an isothiocyanate, a maleimide group, an aliphatic amine, a perfluorobenzamido, an azidoperfluorobenzamido group, or a psoralen. More preferably, RM is a succinimidyl ester of a carboxylic acid, a maleimide, an iodoacetamide, or a reactive platinum complex.

### Conjugated Substances

Preparation of a conjugate of a dye of the disclosure confers the advantageous properties of the dye onto the resulting dye-conjugate. These advantageous properties make the resulting dye-conjugates useful for a wide variety of applications.

Preparation of the desired dye-conjugate begins with the selection of an appropriate reactive compound of the disclosure for the preparation of the desired dye-conjugate. Particularly useful dye-conjugates include, among others, conjugates of peptides, nucleotides, antigens, steroids, vitamins, drugs, haptens, metabolites, toxins, environmental pollutants, amino acids, proteins, nucleic acids, carbohydrates, lipids, ion-complexing moieties, or glass, plastic or other non-biological substances. Alternatively, the conjugated substance is a cell, cellular system, cellular fragment, or subcellular particle, e.g. inter alia, a virus particle, bacterial particle, virus component, biological cell (such as animal cell, plant cell, bacteria, yeast, or protist), or cellular component. Reactive dyes typically label functional groups at the cell surface, in cell membranes, organelles, or cytoplasm.

Typically the conjugated substance is an amino acid, peptide, protein, tyramine, polysaccharide, ion-complexing moiety, nucleoside, nucleotide, oligonucleotide, nucleic acid, hapten, psoralen, drug, hormone, lipid, lipid assembly, polymer, polymeric microparticle, biological cell or virus. More typically, the conjugated substance is a peptide, a protein, a nucleotide, an oligonucleotide, or a nucleic acid.

In another embodiment, the conjugated substance is a biological polymer such as a peptide, protein, oligonucleotide, or nucleic acid polymer that is also labeled with at least a second fluorescent dye (optionally an additional dye of the present disclosure), to form an energy-transfer pair. In some aspects, the labeled conjugate functions as an enzyme substrate, and enzymatic hydrolysis of the substrate disrupts energy transfer between the first and second dyes. Alternatively, the conjugated substance itself is a fluorescent dye (such as green fluorescent proteins and Phycobiliproteins).

In another embodiment, the conjugated substance is an amino acid (including those that are protected or are substituted by phosphates, carbohydrates, or C₁ to C₂₅ carboxylic acids), or is a polymer of amino acids such as a peptide or protein. Preferred conjugates of peptides contain at least five amino acids, more preferably 5 to 36 amino acids. Preferred peptides include, but are not limited to, neuropeptides, cytokines, toxins, protease substrates, and protein kinase substrates. Preferred protein conjugates include enzymes, antibodies, lectins, glycoproteins, histones, albumins, lipoproteins, avidin, streptavidin, protein A, protein G, phycobiliproteins and other fluorescent proteins, hormones, toxins, chemokines and growth factors. In one preferred aspect, the conjugated protein is a phycobiliprotein, such as allophycocyanin, phycocyanin, phycoerythrin and allophycocyanin B (for example, see U.S. Pat. No. 5,714,386 to Roederer (1998); hereby incorporated by reference). Particularly preferred are conjugates of R-phycoerythrin and of allophycocyanin with selected dyes of the disclosure that serve as excited-state energy acceptors or donors. In these conjugates, excited state energy transfer results in long wavelength fluorescence emission when excited at relatively short wavelengths.

In yet another embodiment, the conjugated substance is a nucleic acid base, nucleoside, nucleotide or a nucleic acid polymer, including those that are modified to possess an additional linker or spacer for attachment of the compounds of the disclosure, such as an alkynyl linkage (U.S. Pat. No. 5,047,519), an aminoallyl linkage (U.S. Pat. No. 4,711,955), a heteroatom-substituted linker (U.S. Pat. No. 5,684,142), or other linkage (all hereby incorporated by reference). In another embodiment, the conjugated substance is a nucleoside or nucleotide analog that links a purine or pyrimidine base to a phosphate or polyphosphate moiety through a noncyclic spacer. In another embodiment, the dye is conjugated to the carbohydrate portion of a nucleotide or nucleoside, typically through a hydroxyl group but additionally through a thiol or amino group (U.S. Pat. Nos. 5,659,025, 5,668,268, 5,679,785; all). Typically, the conjugated nucleotide is a nucleoside triphosphate or a deoxynucleoside triphosphate or a dideoxynucleoside triphosphate. Incorporation of methylene moieties or nitrogen or sulfur heteroatoms into the phosphate or polyphosphate moiety is also useful. Nonpurine and nonpyrimidine bases such as 7-deazapurines (U.S. Pat. No. 6,150,510, incorporated by reference) and nucleic acids containing such bases can also be coupled to dyes of the disclosure. Nucleic acid adducts prepared by reaction of depurinated nucleic acids with amine, hydrazide or hydroxylamine derivatives provide an additional means of labeling and detecting nucleic acids, e.g. “A method for detecting abasic sites in living cells: age-dependent changes in base excision repair.” Atamna H, Cheung I, Ames B N. Proc Natl Acad Sci U.S. Pat. No. 97, 686-691 (2000).

Preferred nucleic acid polymer conjugates are labeled, single- or multi-stranded, natural or synthetic DNA or RNA, DNA or RNA oligonucleotides, or DNA/RNA hybrids, or incorporate an unusual linker such as morpholine derivatized phosphates, or peptide nucleic acids such as N-(2-aminoethyl)glycine units. When the nucleic acid is a synthetic oligonucleotide, it typically contains fewer than 50 nucleotides, more typically fewer than 25 nucleotides. Conjugates of peptide nucleic acids (PNA) (Nielsen et al U.S. Pat. No. 5,539,082) may be preferred for some applications because of their generally faster hybridization rates.

In another embodiment, the conjugated oligonucleotides of the disclosure are aptamers for a particular target molecule, such as a metabolite, dye, hapten, or protein. That is, the oligonucleotides have been selected to bind preferentially to the target molecule. Methods of preparing and screening aptamers for a given target molecule have been previously described and are known in the art (for example, U.S. Pat. No. 5,567,588 to Gold (1996); hereby incorporated by reference).

In one embodiment, conjugates of biological polymers such as peptides, proteins, oligonucleotides, nucleic acid polymers are also labeled with at least a second fluorescent dye, which is optionally an additional dye of the present disclosure, to form an energy-transfer pair. In some aspects of the disclosure, the labeled conjugate functions as an enzyme substrate, and enzymatic hydrolysis disrupts the energy transfer. In another embodiment of the disclosure, the energy-transfer pair that incorporates a dye of the disclosure is conjugated to an oligonucleotide that displays efficient fluorescence quenching in its hairpin conformation [the so-called “molecular beacons” of Tyagi et al., NATURE BIOTECHNOLOGY 16, 49 (1998)] or fluorescence energy transfer.

### Selected Embodiments

Selected examples of the compounds of the disclosure are provided in Table 2.

TABLE 2
Selected embodiment of the compounds of the disclosure

| Cpd. | |
|---|---|
| No. | Chemical Structure |
| 1 | 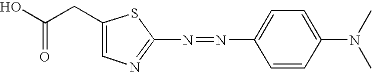 |
| 2 | 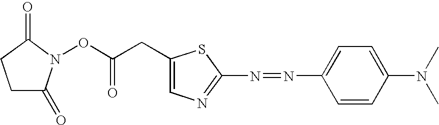 |
| 4 | 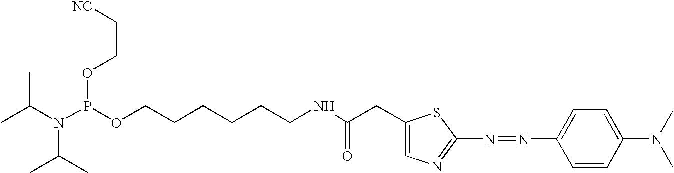 |
| 5 | 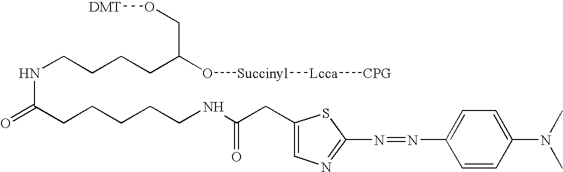 |
| 6 | 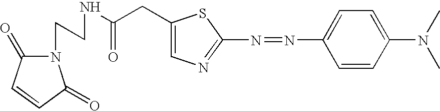 |
| 7 | 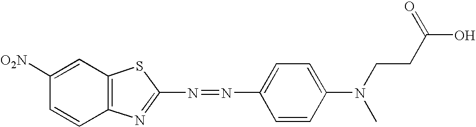 |
| 8 | 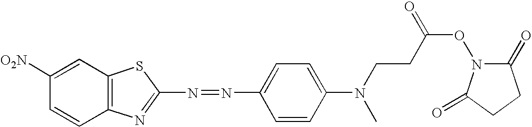 |
| 10 | 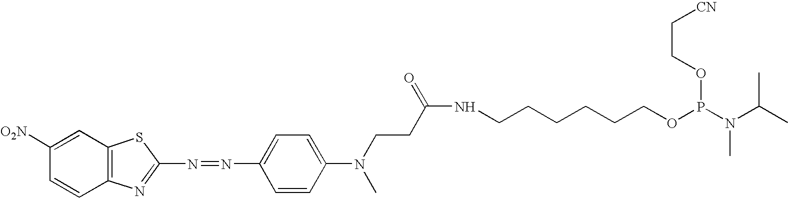 |
| 11 | 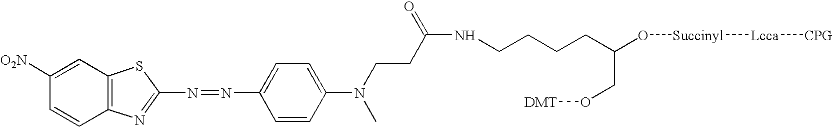 |
| 13 | 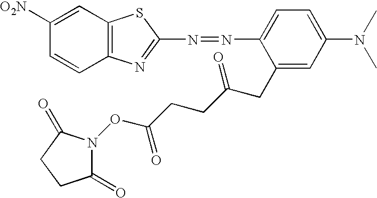 |
| 15 | 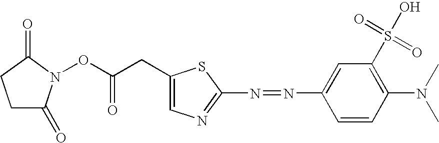 |
| 16 | 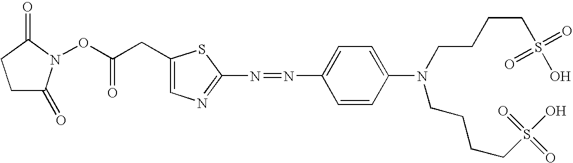 |
| 18 | 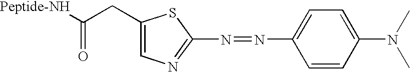 |
| 19 | 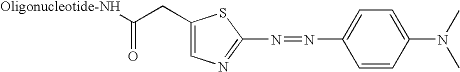 |
| 20 | 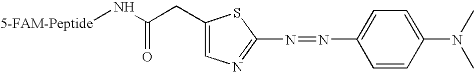 |
| 21 | 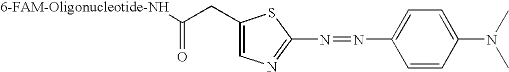 |
| 22 | 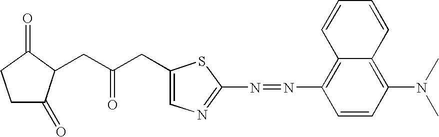 |
| 23A | 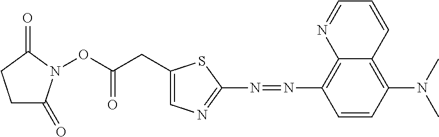 |
| 23 | 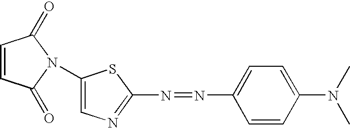 |
| 24 | 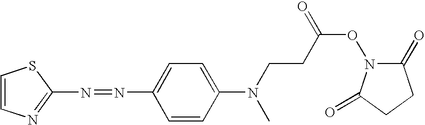 |
| 25 | 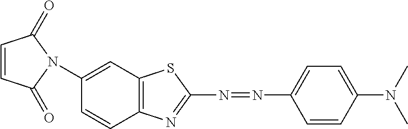 |
| 26 | 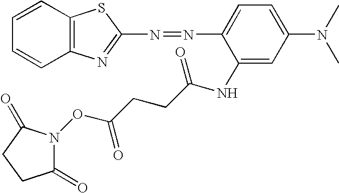 |
| 27 | 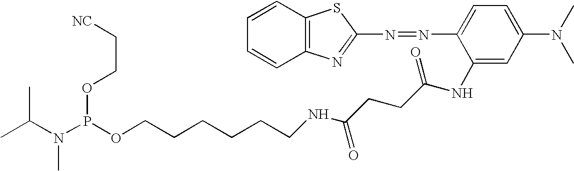 |
| 28 | 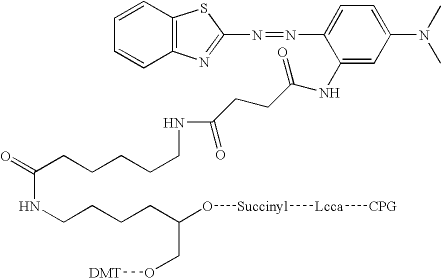 |
| 29 | 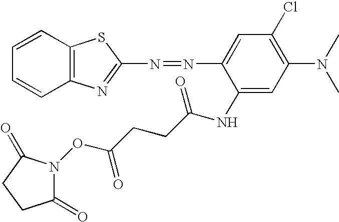 |
| 31 | 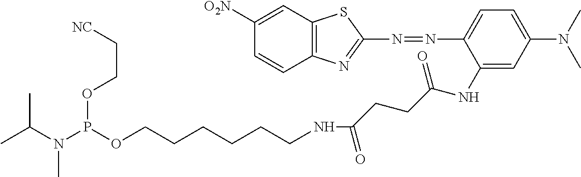 |
| 32 | 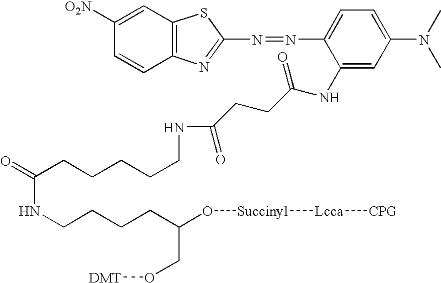 |
| 33 | 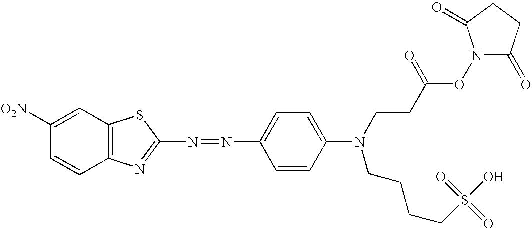 |
| 34 | 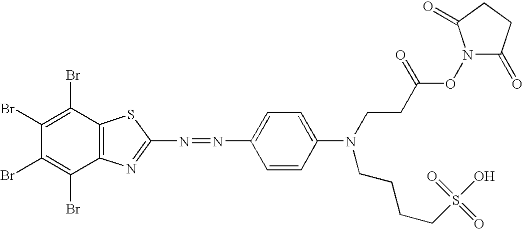 |
| 37 | 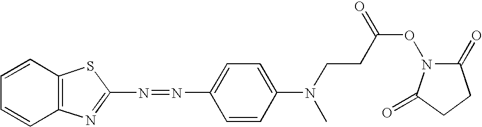 |
| 38 | 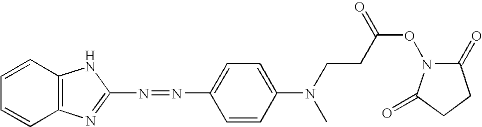 |
| 39 | 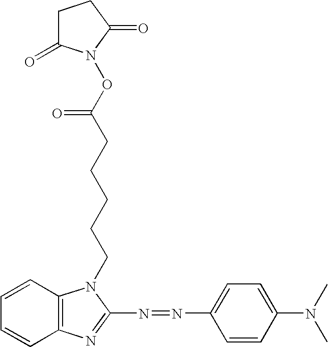 |
| 40 | 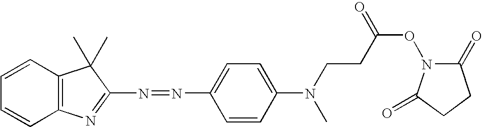 |
| 41 | 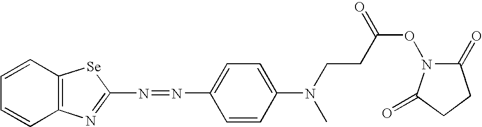 |
| 42 | 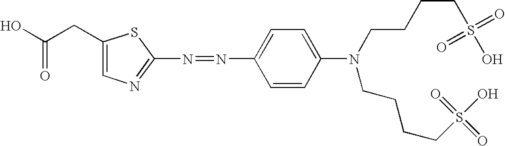 |
| 43 | 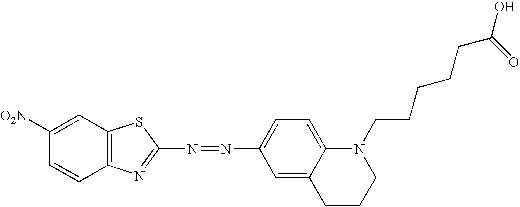 |
| 44 | 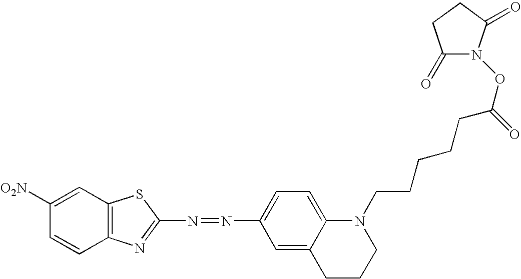 |
| 45 | 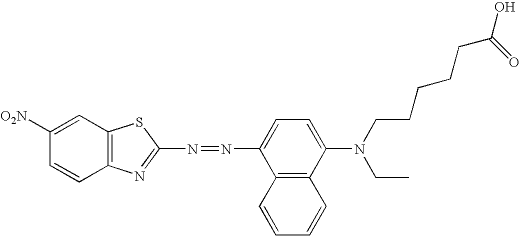 |
| 46 | 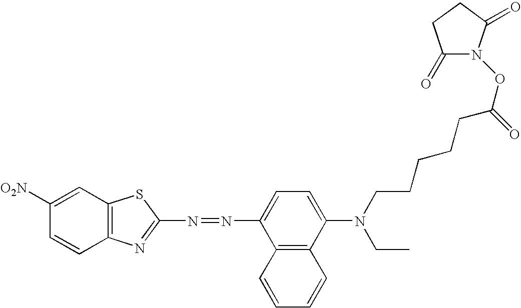 |
| 47 | 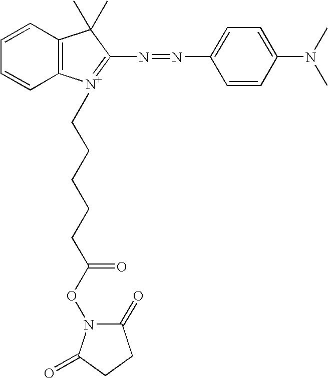 |
| 48 | 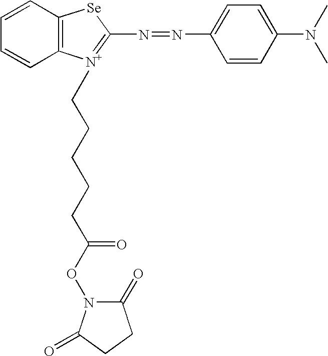 |
| 49 | 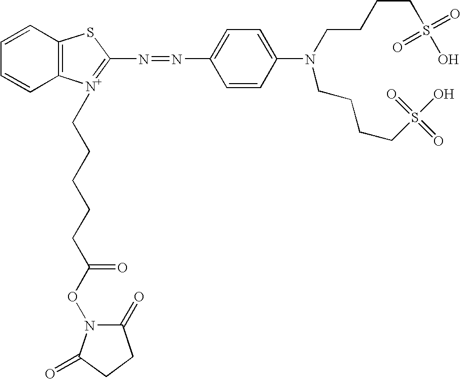 |
| 50 | 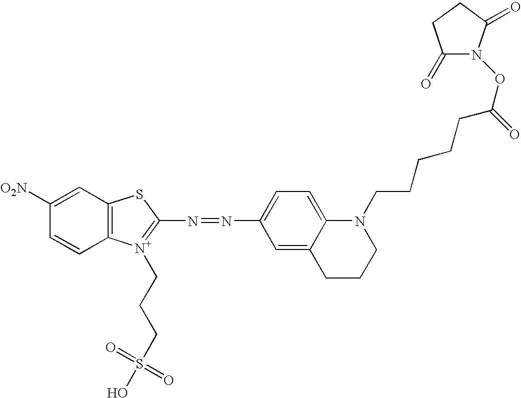 |
| 51 | 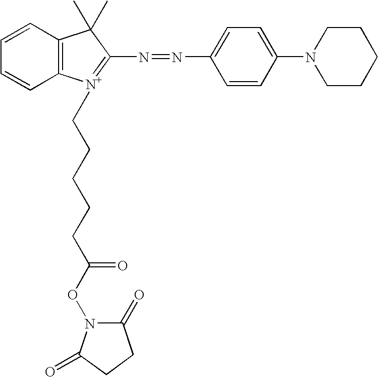 |
| 52 | 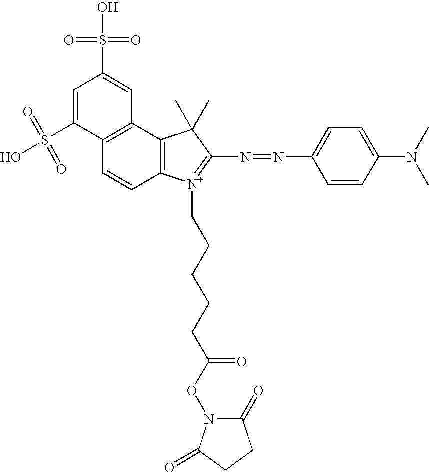 |
| 53 | 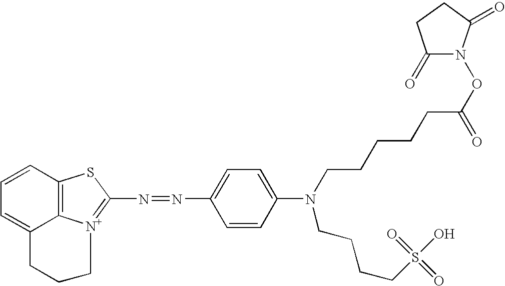 |
| 54 | 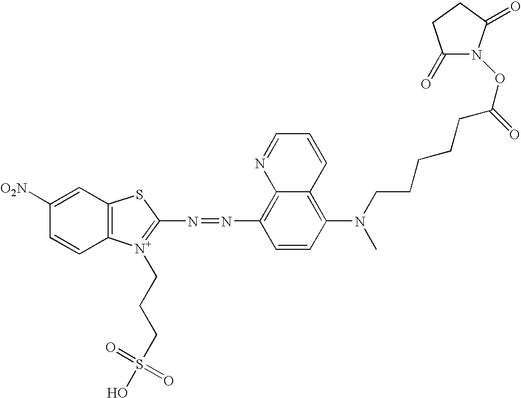 |
| 55 | 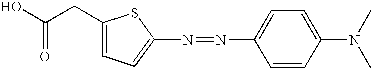 |
| 56 | 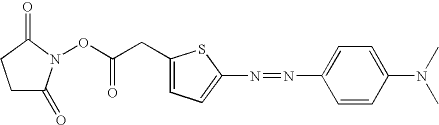 |
| 57 | 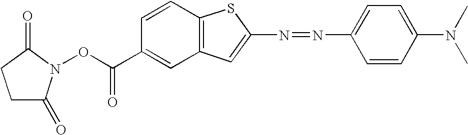 |
| 58 | 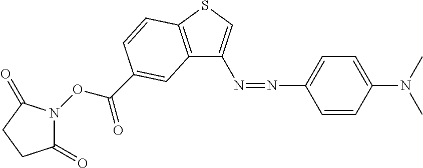 |

The preparation of dye conjugates using chemically reactive dyes is well documented, e.g. by R. Haugland, MOLECULAR PROBES HANDBOOK OF FLUORESCENT PROBES AND RESEARCH CHEMICALS, Chapters 1-3 (1996); and Brinkley, BIOCONJUGATE CHEM., 3, 2 (1992). Conjugates typically result from mixing appropriate reactive dyes and the substance to be conjugated in a suitable solvent, i.e. a solvent in which both components are soluble. The majority of the dyes of the disclosure are readily soluble in aqueous solutions, facilitating conjugation reactions with most biological materials. For those reactive dyes that are photoactivated, conjugation requires illumination of the reaction mixture to activate the reactive dye.

In one embodiment, the compounds of the disclosure may be described by Formula II:

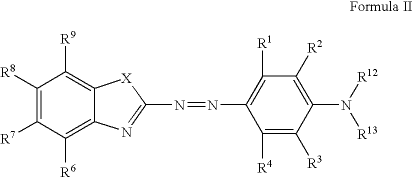

where X is CH₂, CH—R¹⁰, C—R¹⁰R¹¹, O, S, Se, NH or N—R¹⁰.

Dye substituents R¹ to R⁹ are independently hydrogen, cyano, alkyl having from 1-20 carbons, alkoxy having from 1-20 carbons, trifluoromethyl, halogen, methylthio, sulfonyl, phosphonyl, boronyl, carbonyl, hydroxy, amino, thiol, aryl or an RM; R¹⁰ to R¹³ are independently alkyl, polyethylene glycol, aryl or an RM; Alternatively one or more of R¹ and R², R³ and R⁴, R⁶ and R⁷, R⁷ and R⁸, R⁸ and R⁹, or R⁹ and R¹⁰ when taken in combination form an aryl or heteroaryl ring; or one or more of R² and R¹², or R³ and R¹³ taken in combination form a 5- to 8-membered ring; or R¹² and R¹³ when taken in combination form a 3- to 12-membered ring; provided that at least one of R¹ to R¹³ and X is an RM.

In yet another embodiment, the compounds of the disclosure may be described by Formula III:

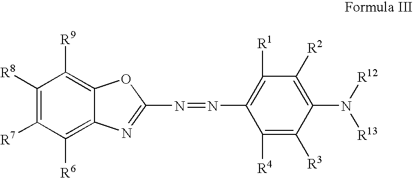

where R¹ to R⁹ are independently hydrogen, alkyl having from 1-20 carbons, alkoxy having from 1-20 carbons, trifluoromethyl, halogen, methylthio, cyano, sulfonyl, phosphonyl, boronyl, carbonyl, hydroxy, amino, thiol, aryl or an RM; R¹⁰ to R¹³ are independently alkyl, polyethylene glycol, aryl or an RM; Alternatively one or more of R¹ and R², R³ and R⁴, R⁶ and R⁷, R⁷ and R⁸, R⁸ and R⁹, or R⁹ and R¹⁰ when taken in combination form an aryl or heteroaryl ring; or one or more of R² and R¹², or R³ and R¹³ taken in combination form a 5- to 8-membered ring; or R¹² and R¹³ when taken in combination form a 3- to 12-membered ring; provided that at least one of R¹ to R¹³ is an RM.

In yet another embodiment, the compounds of the disclosure may be described by Formula IV:

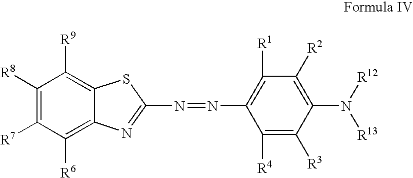

where R¹ to R⁹ are independently hydrogen, alkyl having from 1-20 carbons, alkoxy having from 1-20 carbons, trifluoromethyl, halogen, methylthio, cyano, sulfonyl, phosphonyl, boronyl, carbonyl, hydroxy, amino, thiol, aryl or an RM; R¹⁰ to R¹³ are independently alkyl, polyethylene glycol, aryl or an RM; Alternatively one or more of R¹ and R², R³ and R⁴, R⁶ and R⁷, R⁷ and R⁸, R⁸ and R⁹, or R⁹ and R¹⁰ when taken in combination form an aryl or heteroaryl ring; or one or more of R² and R¹², or R³ and R¹³ taken in combination form a 5- to 8-membered ring; or R¹² and R¹³ when taken in combination form a 3- to 12-membered ring; provided that at least one of R¹ to R¹³ is an RM.

In yet another embodiment, the compounds of the disclosure may be described by Formula V:

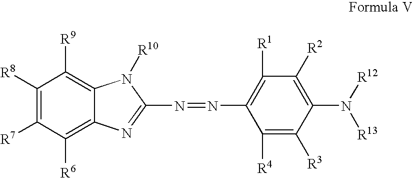

where R¹ to R⁹ are independently hydrogen, alkyl having from 1-20 carbons, alkoxy having from 1-20 carbons, trifluoromethyl, halogen, cyano, methylthio, sulfonyl, phosphonyl, boronyl, carbonyl, hydroxy, amino, thiol, aryl or an RM; R¹⁰ to R¹³ are independently alkyl, polyethylene glycol, aryl or an RM; Alternatively one or more of R¹ and R², R³ and R⁴, R⁶ and R⁷, R⁷ and R⁸, R⁸ and R⁹, or R⁹ and R¹⁰ when taken in combination form an aryl or heteroaryl ring; or one or more of R² and R¹², or R³ and R¹³ taken in combination form a 5- to 8-membered ring; or R¹² and R¹³ when taken in combination form a 3- to 12-membered ring; provided that at least one of R¹ to R¹³ is an RM.

In yet another aspect, the compounds of the disclosure may be described by Formula VI:

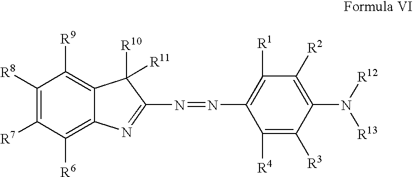

where R¹ to R⁹ are independently hydrogen, alkyl having from 1-20 carbons, alkoxy having from 1-20 carbons, trifluoromethyl, halogen, methylthio, sulfonyl, phosphonyl, boronyl, carbonyl, hydroxy, amino, thiol, aryl or an RM; R¹⁰ to R¹³ are independently alkyl, polyethylene glycol, aryl or an RM; Alternatively one or more of R¹ and R², R³ and R⁴, R⁶ and R⁷, R⁷ and R⁸, R⁸ and R⁹, R⁹ and R¹⁰, or R⁹ and R¹¹ when taken in combination form an aryl or heteroaryl ring; or one or more of R² and R¹², or R³ and R¹³ taken in combination form a 5- to 8-membered ring; or R¹² and R¹³ when taken in combination form a 3- to 12-membered ring; provided that at least one of R¹ to R¹³ is an RM.

In yet another embodiment, the compounds of the disclosure may be described by Formula VII:

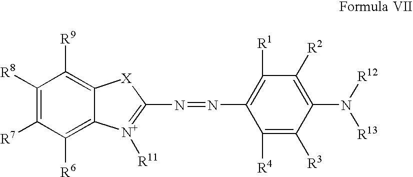

where X is CH₂, CH—R¹⁰, C—R¹⁰R¹¹, O, S, Se, NH or N—R¹⁰

Dye substituents R¹ to R⁹ are independently hydrogen, cyano, alkyl having from 1-20 carbons, alkoxy having from 1-20 carbons, trifluoromethyl, halogen, methylthio, sulfonyl, phosphonyl, boronyl, carbonyl, hydroxy, amino, thiol, aryl or a reactive moiety RM; R¹⁰ to R¹³ are independently alkyl, polyethylene glycol, aryl or an RM; Alternatively one or more of R¹ and R², R³ and R⁴, R⁶ and R⁷, R⁷ and R⁸, R⁸ and R⁹, or R⁹ and R¹⁰ when taken in combination form an aryl or heteroaryl ring; or one or more of R² and R¹², or R³ and R¹³ taken in combination form a 5- to 8-membered ring; or R¹² and R¹³ when taken in combination form a 3- to 12-membered ring; provided that at least one of R¹ to R¹³ is an RM.

In yet another embodiment, the compounds of the disclosure may be described by Formula VIII:

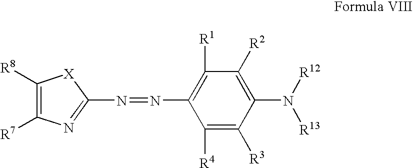

where X is CH₂, CH—R¹⁰, C—R¹⁰R¹¹, O, S, Se, NH or N—R¹⁰. Substituents R¹ to R⁸ are independently hydrogen, alkyl, alkoxy, halogen, alkylthio, cyano, sulfonyl, phosphonyl, boronyl, carbonyl, hydroxy, amino, thiol, aryl, heteroaryl, or a SUBSTRATE where each SUBSTRATE is independently a polymer useful for synthesizing peptides or oligonucleotides; R¹⁰ to R¹³ are independently alkyl, polyethylene glycol, aryl or a SUBSTRATE; Alternatively one or more of R¹ and R², R³ and R⁴, R⁷ and R⁸, or R⁸ and R⁹ when taken in combination form an aryl or heteroaryl ring; or one or more of R² and R¹², or R³ and R¹³ taken in combination form a 5- to 8-membered ring; or R¹² and R¹³ when taken in combination form a 3- to 12-membered ring; provided that at least one of R¹ to R¹³ is a SUBSTRATE.

For this embodiment, at least one of R¹ to R⁸ and X is a conjugated substance that is a SUBSTRATE, where SUBSTRATE is a polymer of molecular weight larger than 1000 daltons, or a compound that responds to a biological stimulus.

In yet another embodiment, the compounds of the disclosure may be described by Formula IX:

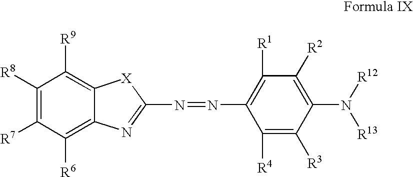

where X is CH₂, CH—R¹⁰, C—R¹⁰R¹¹, O, S, Se, NH or N—R¹⁰. Substituents R¹ to R⁹ are independently hydrogen, alkyl, alkoxy, halogen, alkylthio, cyano, sulfonyl, phosphonyl, boronyl, carbonyl, hydroxy, amino, thiol, aryl, heteroaryl, or a SUBSTRATE where each SUBSTRATE is independently a polymer useful for synthesizing peptides or oligonucleotides; R¹⁰ to R¹³ are independently alkyl, polyethylene glycol, aryl or a SUBSTRATE; Alternatively one or more of R¹ and R², R³ and R⁴, R⁶ and R⁷, R⁷ and R⁸, R⁸ and R⁹, or R⁹ and R¹⁰ when taken in combination form an aryl or heteroaryl ring; or one or more of R² and R¹², or R³ and R¹³ taken in combination form a 5- to 8-membered ring; or R¹² and R¹³ when taken in combination form a 3- to 12-membered ring; provided that at least one of R¹ to R¹³ is a SUBSTRATE.

In yet another embodiment, the compounds of the disclosure may be described by Formula X:

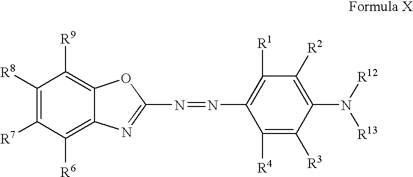

where Substituents R¹ to R⁹ are independently hydrogen, alkyl, alkoxy, halogen, alkylthio, cyano, sulfonyl, phosphonyl, boronyl, carbonyl, hydroxy, amino, thiol, aryl, heteroaryl, or a SUBSTRATE where each SUBSTRATE is independently a polymer useful for synthesizing peptides or oligonucleotides; R¹⁰ to R¹³ are independently alkyl, polyethylene glycol, aryl or a SUBSTRATE; Alternatively one or more of R¹ and R², R³ and R⁴, R⁶ and R⁷, R⁷ and R⁸, R⁸ and R⁹, or R⁹ and R¹⁰ when taken in combination form an aryl or heteroaryl ring; or one or more of R² and R¹², or R³ and R¹³ taken in combination form a 5- to 8-membered ring; or R¹² and R¹³ when taken in combination form a 3- to 12-membered ring; provided that at least one of R¹ to R¹³ is a SUBSTRATE.

In yet another embodiment, the compounds of the disclosure may be described by Formula XI:

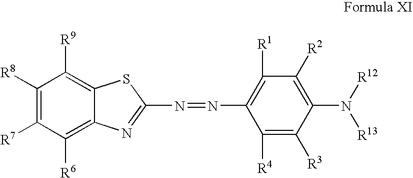

Where Substituents R¹ to R⁹ are independently hydrogen, alkyl, alkoxy, halogen, alkylthio, cyano, sulfonyl, phosphonyl, boronyl, carbonyl, hydroxy, amino, thiol, aryl, heteroaryl, or a SUBSTRATE where each SUBSTRATE is independently a polymer useful for synthesizing peptides or oligonucleotides; R¹⁰ to R¹³ are independently alkyl, polyethylene glycol, aryl or a SUBSTRATE; Alternatively one or more of R¹ and R², R³ and R⁴, R⁶ and R⁷, R⁷ and R⁸, R⁸ and R⁹, or R⁹ and R¹⁰ when taken in combination form an aryl or heteroaryl ring; or one or more of R² and R¹², or R³ and R¹³ taken in combination form a 5- to 8-membered ring; or R¹² and R¹³ when taken in combination form a 3- to 12-membered ring; provided that at least one of R¹ to R¹³ is a SUBSTRATE.

In yet another embodiment, the compounds of the disclosure may be described by Formula XII:

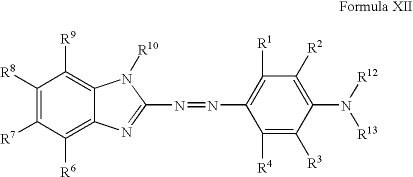

where Substituents R¹ to R⁹ are independently hydrogen, alkyl, alkoxy, halogen, alkylthio, cyano, sulfonyl, phosphonyl, boronyl, carbonyl, hydroxy, amino, thiol, aryl, heteroaryl, or a SUBSTRATE where each SUBSTRATE is independently a polymer useful for synthesizing peptides or oligonucleotides; R¹⁰ to R¹³ are independently alkyl, polyethylene glycol, aryl or a SUBSTRATE; Alternatively one or more of R¹ and R², R³ and R⁴, R⁶ and R⁷, R⁷ and R⁸, R⁸ and R⁹, or R⁹ and R¹⁰ when taken in combination form an aryl or heteroaryl ring; or one or more of R² and R¹², or R³ and R¹³ taken in combination form a 5- to 8-membered ring; or R¹² and R¹³ when taken in combination form a 3- to 12-membered ring; provided that at least one of R¹ to R¹³ is a SUBSTRATE.

In yet another embodiment, the compounds of the disclosure may be described by Formula XIII:

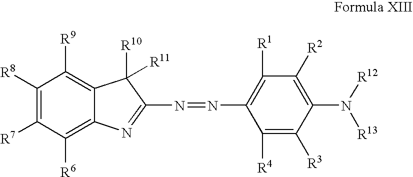

where Substituents R¹ to R⁹ are independently hydrogen, alkyl, alkoxy, halogen, alkylthio, cyano, sulfonyl, phosphonyl, boronyl, carbonyl, hydroxy, amino, thiol, aryl, heteroaryl, or a SUBSTRATE where each SUBSTRATE is independently a polymer useful for synthesizing peptides or oligonucleotides; R¹⁰ to R¹³ are independently alkyl, polyethylene glycol, aryl or a SUBSTRATE; Alternatively one or more of R¹ and R², R³ and R⁴, R⁶ and R⁷, R⁷ and R⁸, R⁸ and R⁹, R⁹ and R¹⁰, or R⁹ and R¹¹ when taken in combination form an aryl or heteroaryl ring; or one or more of R² and R¹², or R³ and R¹³ taken in combination form a 5- to 8-membered ring; or R¹² and R¹³ when taken in combination form a 3- to 12-membered ring; provided that at least one of R¹ to R¹³ is a SUBSTRATE.

In yet another embodiment, the compounds of the disclosure may be described by Formula XIV:

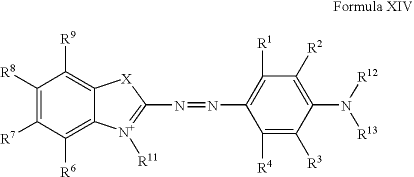

where Substituents R¹ to R⁹ are independently hydrogen, alkyl, alkoxy, halogen, alkylthio, cyano, sulfonyl, phosphonyl, boronyl, carbonyl, hydroxy, amino, thiol, aryl, heteroaryl, or a SUBSTRATE where each SUBSTRATE is independently a polymer useful for synthesizing peptides or oligonucleotides; R¹⁰ to R¹³ are independently alkyl, polyethylene glycol, aryl or a SUBSTRATE; Alternatively one or more of R¹ and R², R³ and R⁴, R⁶ and R⁷, R⁷ and R⁸, R⁸ and R⁹, R⁹ and R¹⁰, or R⁶ and R¹¹ when taken in combination form an aryl or heteroaryl ring; or one or more of R² and R¹², or R³ and R¹³ taken in combination form a 5- to 8-membered ring; or R¹² and R¹³ when taken in combination form a 3- to 12-membered ring; provided that at least one of R¹ to R¹³ is a SUBSTRATE.

### Synthesis

The preparation of azo dyes has been generally described in the chemical literature (see K. Venkataraman, Chemistry of Synthetic Dyes (Organic & Biological Chemistry), Academic Press Inc., New York (1971); H. A. Lubs, The Chemistry of Synthetic Dyes & Pigments, Reinhold Publishing C., New York (1955); W. B. O'Brien, Factory Practice in Manufacture of Azo Dyes, The Chemical Pub. Co., New York (1924)). Azo dyes may be prepared by the coupling of electron-rich aromatics with a diazonium salt that is usually prepared from the corresponding aromatic amine. These basic azo structures are optionally further substituted, during or after synthesis, to give the corresponding dye substituents as defined above. It is recognized that there are many possible variations that may yield an equivalent results.

Dyes which incorporate a reactive functional group, such as those described in Table 1 among others, may be prepared using methods adapted from those documented in the literature. Particularly useful examples are amine-reactive dyes such as activated esters of carboxylic acids, which are typically synthesized by coupling a carboxylic acid to a relatively acidic leaving group. Other preferred amine-reactive groups include sulfonyl halides, which are prepared from sulfonic acids using a halogenating agent such as PCl₅ or POCl₃; halotriazines, which are prepared by the reaction of cyanuric halides with amines; isocyanates or isothiocyanates, which are prepared from amines and phosgene or thiophosgene; and phosphoramidites, which are prepared from alcohols or phenols respectively.

Dyes containing amines and hydrazides are particularly useful for conjugation to carboxylic acids, aldehydes and ketones. Most often these are synthesized by reaction of an activated ester of a carboxylic acid or a sulfonyl halide with a diamine, such as cadaverine, or with a hydrazine. Alternatively, aromatic amines are commonly synthesized by chemical reduction of a nitroaromatic compound. Amines and hydrazines are particularly useful precursors for synthesis of thiol-reactive haloacetamides or maleimides by standard methods.

Dye building blocks containing amino acids such as FMOC-Lys(Dye)-OH, FMOC-Asp(Dye)-OH and FMOC-Glu(Dye) are readily used for the preparation of peptides that contain the compounds of the disclosure using the standard FMOC chemistry in an automated set up [E. Atherton, Sheppard, R. C. Solid Phase peptide synthesis: a practical approach. Oxford, England: IRL Press (1989); J. M. Stewart and J. D. Young, Solid phase peptide synthesis, 2nd ed, Rockford: Pierce Chemical Company (1984).]. Alternatively dye building blocks containing amino acids such as BOC-Lys(Dye)-OH, BOC-Asp(Dye)-OH and BOC-Glu(Dye) are readily used for the preparation of peptides that contain the compounds of the disclosure using the standard BOC chemistry or using liquid phase chemistry [N. Benoiton, Chemistry of Peptide Sythesis (2005); G. Jung, Combinatorial peptide and nonpeptide libraries, A Handbook, VCH: New York (1996)].

Nucleosides and nucleotides labeled with dyes of the disclosure may be particularly useful for some applications of nucleic acid labeling. The use of dye phosphoramidites for labeling nucleotides and nucleosides have been previously described [U.S. Pat. Nos. 7,019,129 and 7,019,312 to Cook et al. (2006); U.S. Pat. No. 5,986,086 to Bruch et al. (1999); U.S. Pat. No. 5,808,044 to Brush et al. (1998); U.S. Pat. No. 5,556,959 to Brush et al. (1996) all hereby incorporated by reference].

Multiple dyes might be conjugated to a single large polymer such as an antibody, a protein, a carbohydrate, a lipid, a nucleic acid or a latex bead using standard bioconjugation techniques, such as those disclosed by R. Haugland, MOLECULAR PROBES HANDBOOK OF FLUORESCENT PROBES AND RESEARCH CHEMICALS, Chapters 1-3 (1996); and Brinkley, BIOCONJUGATE CHEM., 3, 2 (1992). Conjugates typically result from mixing appropriate reactive dyes and the substance to be conjugated in a suitable solvent in which both are soluble. The majority of the dyes of the disclosure are readily soluble in aqueous solutions, facilitating conjugation reactions with most biological materials. For those reactive dyes that are photoactivated, conjugation requires illumination of the reaction mixture to activate the reactive dye.

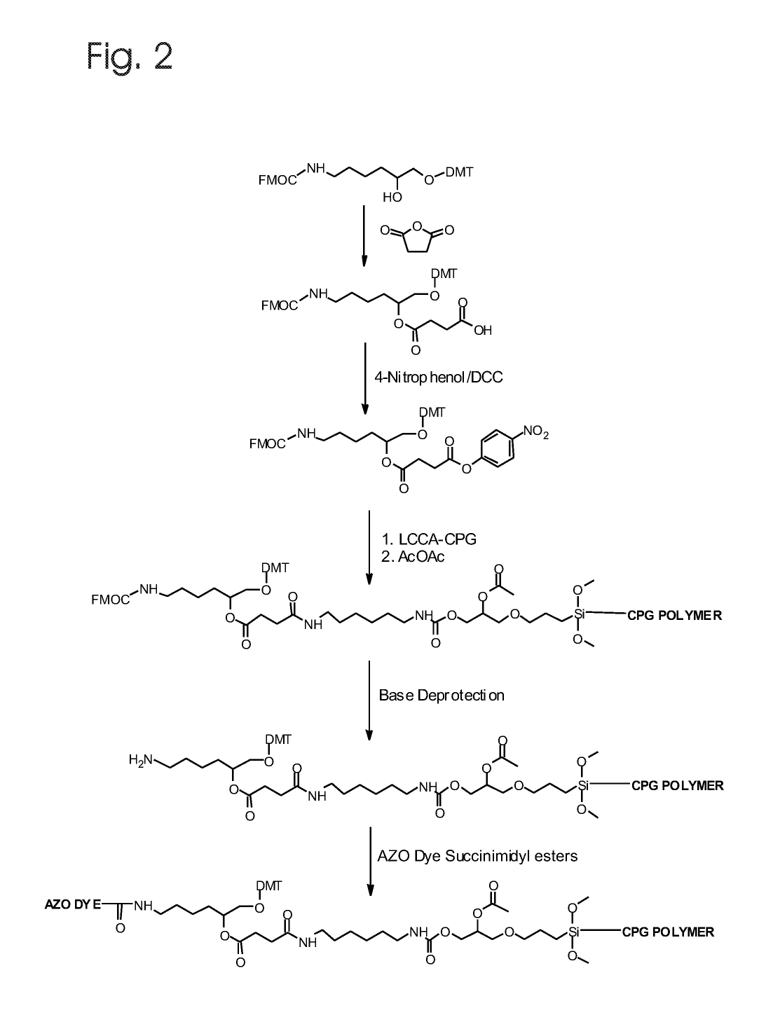

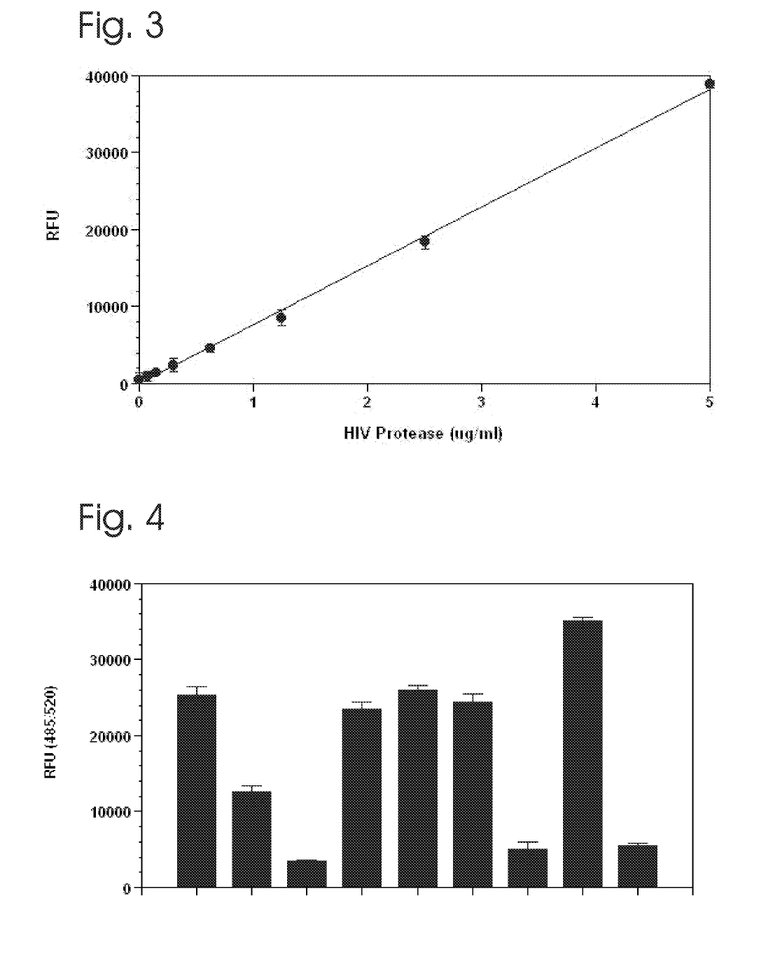

Examples of some synthetic strategies for selected dyes of the disclosure, as well as their characterization, synthetic precursors, conjugates and method of use are provided in the schemes of FIGS. 2 and 3 as well as in the examples below. Further modifications and permutations will be obvious to one skilled in the art.

### APPLICATIONS AND METHODS OF USE

The compounds of the disclosure are useful as components in a variety of biological applications. For example, the dyes of the disclosure may be used as colorimetric labels for a conjugated substance. The compounds of the disclosure typically exhibit large extinction coefficients, and thereby permit the detection of the quenching compound-conjugated substance by virtue of visible light absorption of the conjugated compound.

The compounds of the disclosure are typically able to accept energy transfer from a wide variety of fluorophores. in this application, the compounds of the disclosure may be referred to as quenching compounds. Energy transfer may occur provided that the quenching compound and the fluorophore are in sufficiently close proximity for quenching to occur, and that at least some spectral overlap occurs between the emission wavelengths of the fluorophore and the absorption band of the quenching compound. This overlap may occur with emission of the donor occurring at a lower or even higher wavelength emission maximum than the maximal absorbance wavelength of the quenching compound, provided that sufficient spectral overlap exists. Energy transfer may also occur through transfer of emission of the donor to higher electronic states of the acceptor, such as from tryptophan residues of proteins to the weaker absorption bands between 300 and 350 nm typical of the dyes in the ultraviolet region. Preferably, the quenching compound of the disclosure is only dimly fluorescent, or substantially nonfluorescent, so that energy transfer results in little or no fluorescence emission. In one aspect of the disclosure, the quenching compound of the disclosure has a fluorescence quantum yield of less than about 0.1. In another aspect of the disclosure, the quenching compound has a fluorescence quantum yield of less than about 0.05. In yet another aspect of the disclosure, the quenching compound has a fluorescence quantum yield of less than about 0.01

Typically, such fluorescence quenching occurs through Fluorescence Resonance Energy Transfer (FRET) between a donor and a quenching compound of the disclosure acting as an energy acceptor. The degree of FRET exhibited by a donor-acceptor pair can be represented by the equations [1] to [3]:

The efficiency (E%) and rate (k_{T}) of FRET are respectively expressed as follows:

E%=k_{T}/(τ_{D}⁻¹+k_{T}) [1]

k_{T}=Rₒ⁶γ⁻⁶τ_{D}⁻¹ [2]

Where τ_{D} is the decay time of the donor in the absence of acceptor; γ is the donor-acceptor (D-A) distance; Rₒ is the Förster distance where FRET has 50% efficiency, is typically in the range of 20-60 Å. Rₒ is determined by the following equation:

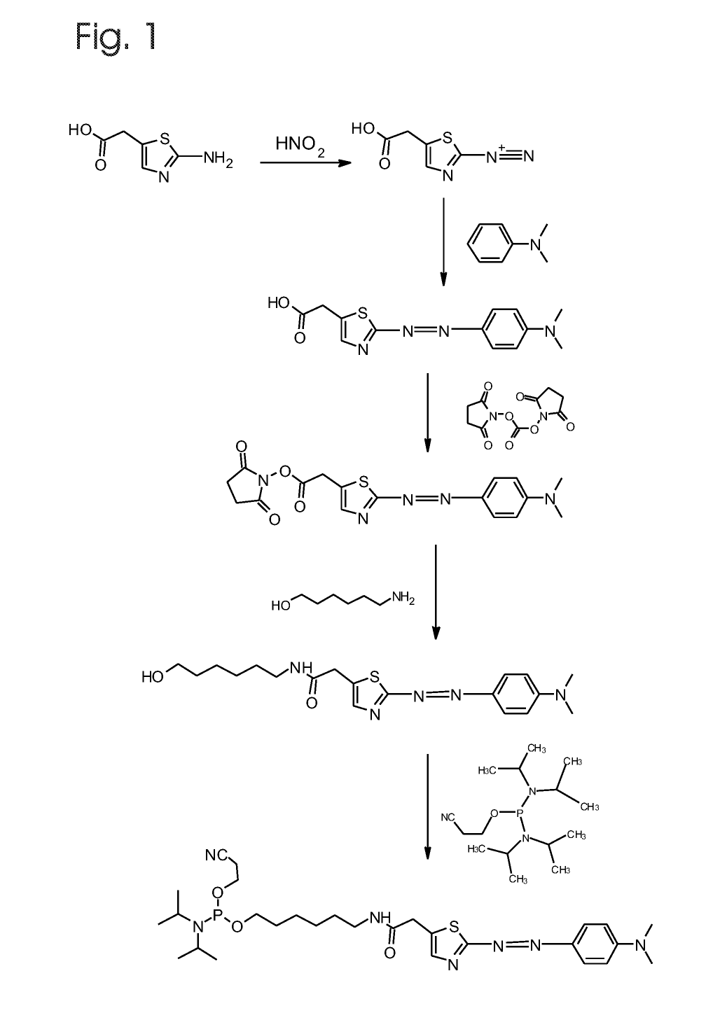

Rₒ⁶=8.79×10²³[k²n⁻⁴Φ_{D}J(λ)] [3]

Where k² is dipole-dipole orientation factor (ranging from 0 to 4, k²=2/3 for randomly oriented donors and acceptors); n is refractive index [The refractive index is generally known from solvent composition or estimated for macromolecules such as proteins and nucleic acids. n is often assumed to be that of water (n=1.33) for aqueous solutions, or to be that of small organic molecules (n=1.39) for organic solutions]. Φ_{D} is the fluorescence quantum yield of donor in the absence of acceptor. J(λ) is FRET spectral overlap integral as illustrate in FIG. 1, and is determined by the following equation:

J(λ)=∫F_{D}(λ)ε_{A}(λ)λ⁴d(λ) [4]

Where F_{D}(λ) is the corrected fluorescence intensity of the donor in the wavelength range λ to λ+Δλ with the total intensity (area under the curve) normalized unity; ε_{A} is extinction coefficient of the acceptor at λ.

From the above equations, it is easily concluded that for the most efficient FRET the donor-acceptor pair should satisfy the following criteria:
Distance between donor and acceptor: Donor and acceptor molecules must be in close proximity (typically 10-100 Å).
Spectral overlap: The absorption spectrum of the acceptor must overlap fluorescence emission spectrum of the donor.
Dipole orientation: Donor and acceptor transition dipole orientations must be approximately parallel.

It should be readily appreciated that the degree of energy transfer during FRET, and therefore quenching, is highly dependent upon the separation distance between the fluorophore and the quenching compound. In molecular systems, a change in fluorescence quenching typically correlates well with a change in the separation distance between the fluorophore molecules and the quenching compound molecules. Assays that detect such changes in fluorescence are therefore useful for the detection of a great many structural changes, such as changes in molecular conformation, assembly of structures, or degradation of structures.

Any fluorophore with sufficient spectral overlap with a quenching compound of the disclosure, as calculated above, is a suitable donor for FRET applications, other factors being equal. The greater the degree of overlap, the greater the overall quenching observed. While fluorescent dyes are preferred for energy transfer applications, any emission that generates light having sufficient spectral overlap with the quenching compounds of the disclosure is also useful, such as chemifluorescence, or phosphorescence, whether by FRET or by triplet state to singlet state transfer.

While FRET is the most common mechanism for quenching of fluorescence to occur, any combination of molecular orientation and spectral coincidence that results in quenching of fluorescence is a useful mechanism for quenching by the quenching compounds of the disclosure, as described herein. For example, efficient quenching can occur even in the absence of spectral overlap if the fluorophore and the quenching compound are sufficiently close together to form a ground-state complex (as described in Tyagi et al., NATURE BIOTECHNOLOGY 16, 49 (1998)).

Typically, the donor-acceptor includes a quenching compound of the disclosure and a fluorophore that is a fluorescent aromatic or heteroaromatic compound that is a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a azo, a carbocyanine, a carbostyryl, a porphyrin, a salicylate, an anthranilate, an azulene, a perylene, a pyridine, a quinoline, a coumarin, a polyazaindacene [such as 4-bora-3a,4a-diaza-s-indacene as described in U.S. Pat. No. 4,774,339 to Haugland, et al. (1988); U.S. Pat. No. 5,187,288 to Kang, et al. (1993); U.S. Pat. No. 5,248,782 to Haugland, et al. (1993); U.S. Pat. No. 5,274,113 to Kang, et al. (1993); and U.S. Pat. No. 5,433,896 to Kang, et al. (1995)], a azo, an oxazine or a benzoxazine, a carbazine [U.S. Pat. No. 4,810,636 to Corey (1989)], or a phenalenone or benzphenalenone [U.S. Pat. No. 4,812,409 to Babb et al. (1989)] or a nanocrystal. The donor dye is optionally an organic molecule that is a fluorophore, or a fluorescent protein such as a phycobiliprotein or “green fluorescent protein” or a nanocrystal. Preferably, the donor dye is a carbazine, an oxazine, a coumarin, a pyrene, a cyanine, a naphthalene, a phenalenone, or a 4-bora-3a,4a-diaza-s-indacene or a nanocrystal. As used herein, oxazines include resorufins, aminooxazinones, diaminooxazines, and their benzo-substituted analogs. Preferred chemifluorescent dyes include luminol, isoluminol, luciferin, an acridinium ester, or a dioxetane (all cited patents hereby incorporated by reference).

Where the fluorophore is a cyanine [U.S. Pat. Nos. 6,048,982 (2000); 6,133,445 (2001) and 6,686,145 (2004) to Waggoner et al], the synthetic dye is optionally a fluorescein, a rhodol [U.S. Pat. No. 5,227,487 to Haugland, et al. (1993)], or a rhodamine. As used herein, fluorescein includes benzo- or dibenzofluoresceins, seminaphthofluoresceins, or naphthofluoresceins. Similarly, as used herein rhodol includes seminaphthorhodafluors [U.S. Pat. No. 4,945,171 to Haugland, et al. (1990)]. Sulfonated pyrenes, coumarins, carbocyanines, and cyanine dyes have been described previously [U.S. Pat. No. 5,132,432 to Haugland et al., (1992); U.S. Pat. No. 5,696,157 to Wang et al. (1997); U.S. Pat. No. 5,268,486 to Waggoner et al. (1993) all cited patents hereby incorporated by reference].

The quenching compounds of the disclosure are useful in any application where energy transfer from a fluorescent donor to a non-fluorescent acceptor has previously been described, provided that some spectral overlap exists between the emission of the donor dye and the absorbance of the quenching compound of the disclosure. Typically, the quenching compounds are used in combination with a fluorophore in a method that detects a change in separation distance between the fluorophore and the quenching compound.

The donor fluorophores and quenching compounds used in the instant methods are useful in any medium in which they are sufficiently soluble. For example, selected embodiments of the instant quenching compounds that are substituted by highly non-polar substituents may be useful in organic solvents, or on or in non-polar matrices, such as polymeric microspheres. For biological applications, the quenching compounds of the disclosure and their conjugates are typically used in an aqueous, mostly aqueous or aqueous-miscible solution prepared according to methods generally known in the art.

Chemically reactive compounds of the disclosure may covalently attach to a corresponding functional group on a wide variety of materials, forming conjugates as described above. Photoreactive compounds of the disclosure can be used similarly to photolabel nucleic acids, or components of the outer membrane of biological cells, or as photo-fixable polar tracers for cells.

The quenching compounds of the disclosure are generally utilized by labeling a substance or sample of interest under conditions selected so that illumination of the sample with an appropriate wavelength of light results in a detectable optical response. In one embodiment, the quenching compounds of the disclosure are utilized as colorimetric labels, such that the detectable optical response is an absorption of illumination energy. In another embodiment the quenching compound accepts energy from a donor, such that the detectable optical response is quenching of the fluorescence of the donor.

In most applications of the instant compounds, the labeled substance is utilized in a homogenous solution assay, where specific spatial resolution is not required. In these embodiments of the disclosure the loss of, or restoration of, fluorescence in the sample is detected. In another embodiment, the quenching compound forms a covalent or non-covalent association or complex with an element of the sample where a fluorescent component is present or is subsequently added. In this embodiment, illumination of the sample reveals either a fluorescence response if quenching is not occurring or the degree of quenching may be observed and correlated with a characteristic of the sample. Such correlation typically occurs by comparison with a standard or a calibration curve. Typically, a stained sample is illuminated and observed in order to determine a specified characteristic of the sample by comparing the degree of quenching exhibited to a fluorescence standard of determined intensity. The fluorescence standard may be a fluorescent dye such as the fluorophore used to prepare the quenching compound-fluorophore labeled substance, a fluorescent particle (including fluorescent microspheres), a calibration curve prepared by assaying the doubly labeled substance with a known amount of enzyme or degradation activity, or any other standard that can be used to calibrate fluorescence signal intensity as well known in the art.

Typically, the compounds of the disclosure are used in applications that yield information as to the separation distance between one or more fluorophore donors and quenching compound acceptors. These applications typically include the steps of illuminating the sample under study; detecting the fluorescence response of the system; exposing the sample to an environmental condition sufficient to change the separation distance, or thought to be sufficient to change the separation distance, illuminating the molecular system again, detecting the fluorescence response of the molecular system again, and comparing the first detected fluorescence response to the second detected fluorescence response. The difference in the detected fluorescence before and after the exposure to the selected environmental condition can then be correlated with any change that has occurred in the separation distance between the fluorophores and the quenching compounds, and thereby correlated with an effect of the selected environmental condition.

In one embodiment, the compounds of the disclosure are used in an application that includes preparing a sample that contains a dye-conjugate having the formula

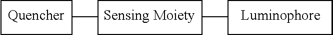

where the Quencher portion of the dye-conjugate is derived from a compound according to any one of Formulas I, II, III, IV, and V; the Luminophore is a luminescent dye; and the Sensing Moiety is a substance selected to be capable of responding to a preselected environmental condition by changing the separation distance between the Quencher and the Luminophore.

The application or assay then includes detecting a first luminescence response of the sample; exposing the sample to an experimental environmental condition that is sufficient to change, or thought to be sufficient to change, the separation distance between the quencher and the Luminophore; detecting a second luminescence response of said sample; determining a difference between the first and second luminescence responses; correlating the difference to a change in the separation distance between the Quencher and the Luminophore; and correlating the change in the separation distance between said Quencher and said Luminophore with the experimental environmental condition.

As discussed in greater detail below, the environmental conditions under which the instant method may be practiced may include the presence or absence of a particular enzyme, the presence or absence of a complementary specific binding pair member, a change in pH, or a change in sample temperature, among other conditions.

### Illumination and Detection

Typically, changes in fluorescence quenching may be detected by methods known in the art for standard fluorescence assays. The fluorescence of a sample, if present, is typically detected by illumination of the sample with a light source capable of producing light that is absorbed at or near the wavelength of maximum absorption of the donor dye, and fluorescence is detected at a wavelength longer than the excitation wavelength, typically near the emission maximum. Such illumination sources include, but are not limited to, hand-held ultraviolet lamps, mercury-arc lamps, xenon lamps, lasers and laser diodes. These illumination sources are optionally integrated into laser scanners, fluorescence microplate readers, standard or minifluorometers, or chromatographic detectors.

The optical response is optionally detected by visual inspection, or by use of instrumentation, including CCD cameras, video cameras, photographic film, laser-scanning devices, fluorometers, photodiodes, quantum counters, epifluorescence microscopes, scanning microscopes, flow cytometers, fluorescence microplate readers, among others. The optical response may be detected directly, or by virtue of a means of amplifying the signal, such as a photomultiplier tube. Where the sample is examined using a flow cytometer, examination of the sample optionally includes sorting portions of the sample according to their fluorescence response.

In the case of a sample in which the labeled substance is immobilized or partially immobilized on a solid or semi-solid support or in a matrix such as agar, sample fluorescence is typically detected using a transilluminator, an epi-illuminator, a laser scanner, a microscope or a similar apparatus that permits observation of the matrix.

Fluorescence occurring within a cell is typically detected using instrumentation that is capable of detecting fluorescent emission in single cells, such as a microscope or a flow cytometer (optionally further being followed by sorting of fluorescent cells). Alternatively, multiple cells may be suspended and fluorescence changes measured as for an assay done in true solution. As described above, the method of the instant disclosure is typically useful for detection of changes in separation distance between a fluorophore donor and a quenching compound acceptor.

Any assay that relies upon the measurement of the proximity of fluorophores and quenching compounds in a system may be carried out using the method of the instant disclosure. The method of the instant disclosure is typically utilized to detect and/or quantify the convergence or divergence of the fluorophore donor and quenching compound acceptor. By convergence is meant a decrease in the average separation distance between the fluorophore and the quenching compound. By divergence is meant an increase in the average separation distance between the fluorophore and the quenching compound.

In one embodiment, the method of the instant disclosure is utilized to detect molecular or structural assembly (convergence). In another embodiment, the method of the disclosure is utilized to detect molecular or structural disassembly (divergence). In yet another embodiment, the method of the disclosure is utilized to detect a conformation change in a molecule, macromolecule or structure (optionally convergence or divergence). In yet another embodiment, the method of the instant disclosure incorporates aspects of the detection of assembly, disassembly, and/or conformation changes.

### Detection of Structural Assembly

In one embodiment, the fluorescence of a fluorophore becomes quenched upon being placed in close proximity to a quenching compound of the disclosure (thereby decreasing the separation distance). The following systems, among others, can be analyzed using energy transfer pairs to detect and/or quantify structural assembly by measuring convergence of the donor and acceptor:

a) protein subunit assembly

b) enzyme-mediated protein assembly

c) molecular dimensions of proteins

d) membrane-protein interactions

e) protein-protein interactions

f) protein-protein-nucleic acid complex assembly

g) receptor/ligand interactions

h) immunoassays

i) nucleic acid hybridization

k) quantitative detection of specific DNA sequence amplification

l) detection of DNA duplex winding

m) nucleic acid-protein interactions

n) nucleic acid-drug interactions

o) primer extension assays for mutation detection

p) reverse transcriptase assay

q) strand exchange in DNA recombination reactions

r) membrane fusion assays

s) transmembrane potential sensing

t) ligation assays

In particular, specific binding pair members labeled with a quenching compound are typically used as probes for the complementary member of that specific binding pair, by methods known in the art. The complementary member is typically labeled with a fluorescent label, and association of the two members of the specific binding pair results in fluorescence quenching. This assay is particularly useful in nucleic acid hybridization assays, evaluation of protein-nucleic acid interaction, and in selected standard immunoassays. In one embodiment, a loss of fluorescence indicates the association of an enzyme with an enzyme substrate, agonist or antagonist, such that the fluorophore on one is brought into close proximity to a quenching compound on the other. Selected preferred specific binding pair members are proteins that bind non-covalently to low molecular weight ligands (including biotin), oligonucleotides, and drug-haptens. Representative specific binding pairs are shown in Table 3.

TABLE 3
Representative Specific Binding Pairs

| antigen | Antibody |
|---|---|
| biotin | avidin, streptavidin, anti-biotin |
| folate | folate-binding protein |
| IgG* | protein A or protein G |
| drug | drug receptor |
| toxin | toxin receptor |
| carbohydrate | lectin or carbohydrate receptor |
| peptide | peptide receptor |
| protein | protein receptor |
| peptide nucleic acid | complementary strand |
| enzyme substrate | Enzyme |
| DNA (RNA) | cDNA (cRNA) |
| hormone | hormone receptor |
| ion | Chelator |

*IgG is an immunoglobulin; cDNA and cRNA are complementary strands used for hybridization

Alternatively, a monomer, labeled with a quenching compound, is incorporated into a polymer labeled with a fluorophore, resulting in quenching of fluorescence. In particular, a quenching compound-labeled nucleotide can be incorporated via the polymerase chain reaction into a double stranded DNA molecular that is labeled with a fluorophore.

### Detection of Structural Disassembly

In another embodiment of the method of the disclosure, the disassembly, cleavage or other degradation of a molecular structure is detected by observing the partial or complete restoration of fluorescence of a fluorophore donor. Typically, the initially quenched fluorescence of a fluorophore associated with the structure becomes dequenched upon being released from the constraint of being in close proximity to a quenching compound of the disclosure. The quenching compound is optionally associated with the same molecular structure as the fluorophore, or the donor and acceptor are associated with adjacent but distinct subunits of the structure. The following systems, among others, can be analyzed using energy transfer pairs to detect and/or quantify structural disassembly:

a). detection of protease activity using fluorogenic substrates (for example MMP protease assays)

b). detection of enzyme-mediated protein modification (e.g. cleavage of carbohydrates/fatty acids, phosphates, prosthetic groups)

c). immunoassays (via displacement/competitive assays)

d). detection of DNA duplex unwinding (e.g. helicase/topoisomerase/gyrase assays)

e). nucleic acid strand displacement

f). dsDNA melting

g). nuclease activity

h). lipid distribution and transport

i). TAQMAN assays

Structure disassembly is typically detected by observing the partial or complete restoration of fluorescence, as a conjugated substance is exposed to a degradation conditions of interest for a period of time sufficient for degradation to occur. A restoration of fluorescence indicates an increase in separation distance between the fluorophore and quenching compound, and therefore a degradation of the conjugated substance. If the detectable difference in fluorescence is detected as the degradation proceeds, the assay is a continuous assay. Since most enzymes show some selectivity among substrates, and as that selectivity can be demonstrated by determining the kinetic differences in their hydrolytic rates, rapid testing for the presence and activity of the target enzyme is provided by the enhancement of fluorescence of the labeled substrate following separation from the quenching compound.

In another embodiment of the disclosure, a single-stranded oligonucleotide signal primer is labeled with both a quenching compound and a fluorescent donor dye, and incorporates a restriction endonuclease recognition site located between the donor dye and the quenching compound. The single-stranded oligonucleotide is not cleavable by a restriction endonuclease enzyme, but upon binding to a complementary (target) nucleic acid, the resulting double stranded nucleic acid is cleaved by the enzyme and the decreased quenching is used to detect the presence of the complementary nucleic acid [U.S. Pat. No. 5,846,726 to Nadeau et al., (1998) incorporated by reference].

A single nucleotide polymorphism (SNP) can be detected through the use of sequence specific primers, by detection of melt temperatures of the double stranded nucleic acid. In this aspect, the complementary or substantially complementary strands are labeled with a quenching compound and a fluorophore donor, respectively, and dissociation of the two strands (melting) is detected by the restoration of fluorescence of the donor.

In yet another example of a divergence assay, the rupture of a vesicle containing a highly concentrated solution of fluorophores and quenching compounds is readily detected by the restoration of fluorescence after the vesicle contents have been diluted sufficiently to minimize quenching.

### Detection of Conformation Changes

In this embodiment, the quenching compound and the fluorescent donor are present on the same or different substances, and a change in the three-dimensional structural conformation of one or more components of the assay results in either fluorescence quenching or restoration of fluorescence, typically by substantially decreasing or increasing the separation distance between the quenching compound and a fluorophore. The following systems, among others, can be analyzed using energy transfer pairs to detect and/or quantify conformation changes:

a). protein conformational changes

b). protein folding

c). structure and conformation of nucleic acids

d). drug delivery

e). antisense oligonucleotides

f). cell-cell fusion (e.g. via the diffusion apart of an initial donor-quenching compound pair)

By conformation change is meant, for example, a change in conformation for an oligonucleotide upon binding to a complementary nucleic acid strand. In one such assay, labeled oligonucleotides are substantially quenched when in solution, but upon binding to a complementary strand of nucleic acid become highly fluorescent [so-called “Molecular Beacons”, as described in European patent application EP 0 745 690, by Tyagi et al (1996)]. Another example detects the change in conformation when an oligonucleotide that has been labeled at its ends with a quenching compound and a fluorophore, respectively, loses its G-quartet conformation upon hybridization to a complementary sequence, resulting in decreased fluorescence quenching [U.S. Pat. No. 5,691,145 to Pitner et al. (1997) incorporated by reference]. Alternatively, the binding of an enzyme substrate within the active site of a labeled enzyme may result in a change in tertiary or quaternary structure of the enzyme, with restoration or quenching of fluorescence.

### Additional Detection Reagents and Assay Kits

When used in complex systems, especially in biological cells, the assays of the instant disclosure are optionally combined with the use of one or more additional detection reagents, such as an antibody, or a stain for another component of the system such as a nucleic acid stain, an organelle stain, a metal ion indicator, or a probe to assess viability of the cell. The additional detection reagent is optionally a fluorescent reagent exhibiting a color that contrasts with the donor dye present in the assay, or is a label that is detectable by other optical or non-optical properties.

One aspect of the instant disclosure is the formulation of kits that facilitate the practice of the methods of the disclosure, as described above. The kits of the disclosure comprises a quenching compound of the disclosure, or colorless quenching compound precursor of the disclosure, typically present conjugated to a nucleotide, oligonucleotide, nucleic acid polymer, peptide, or protein. Typically, the kits further comprise one or more buffering agents, typically present as an aqueous solution. The kits of the disclosure optionally further comprise additional detection reagents, a purification medium for purifying the resulting labeled substance, fluorescence standards, enzymes, enzyme inhibitors, organic solvent, or instructions for carrying out an assay of the disclosure.

In one embodiment, the kits comprise a quenching compound of the disclosure and a fluorescent donor. The quenching compound and fluorescent donor are optionally each attached to a conjugated substance, or present in solution as free compounds. Such a kit would be useful for the detection of cell-cell fusion, as fusion of a cell containing the quenching compound with a cell containing a fluorescent donor would result in quenching of fluorescence. Conjugation of either the quenching compound or the fluorescent donor or both to biomolecules, such as polysaccharides, would help retain the reagents in their respective cells until cell fusion occurred.

In another embodiment, the kits comprise a quenching compound and a fluorescent donor, each conjugated to a complementary member of a specific binding pair. In this aspect of the disclosure, binding of the two specific binding pair members results in quenching of fluorescence, and the kit is useful for the detection of competitive binding to one or the other specific binding pair members, or for the detection of an environmental condition or component that either facilitates or inhibits binding of the specific binding pair members.

In another embodiment, the kits comprise a conjugate of a quenching compound and a conjugate of a fluorescent donor, wherein the conjugates are selected such that the action of a particular enzyme results in covalent or noncovalent association of the two conjugates, resulting in quenching of fluorescence. Where the conjugated substances are nucleotides, oligonucleotides or nucleic acid polymers, the kit is useful for the detection of, for example, ligase, telomerase, helicase, topoisomerase, gyrase, DNA/RNA polymerase, or reverse transcriptase enzymes.

In another embodiment, the kits comprise a biomolecule that is covalently labeled by both a quenching compound of the disclosure and a fluorescent donor. In one aspect, the labeled biomolecule exhibits fluorescence until a specified environmental condition (such as the presence of a complementary specific binding pair) causes a conformation change in the biomolecule, resulting in the quenching of fluorescence. Alternatively, the biomolecule is initially quenched, and a specified environmental condition (such as the presence of an appropriate enzyme or chemical compound) results in degradation of the biomolecule and restoration of fluorescence. Such a kit would be useful for the detection of complementary oligonucleotide sequences (as for MOLECULAR BEACONS™), or for the detection of enzymes such as nuclease, lipase, protease, or cellulase.

The examples provided below are given so as to illustrate the practice of this disclosure. They are not intended to limit or define the scope of the disclosure.

### Example 1

### Preparation of Compound 1

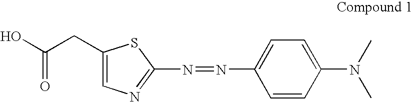

In a beaker, (0.1 mole) of 2-amino-4-carboxymethylthiazole is made into a thin paste with 300 mL of water and dissolved by the addition of concentrated hydrochloric acid (0.11 mole). The solution is cooled to 10° C. with ice, mechanical agitation is started, and more concentrated hydrochloric acid is added (0.11 mole). The temperature being kept at 10-15° C., a solution of sodium nitrite (0.09 mole) in 18 mL of water is added rapidly, and the additional solution of sodium nitrite (0.01 mole) in 2 mL of water is added slowly as needed to give a distinct positive test for nitrous acid on starch-iodide paper. This excess is maintained for one-half hour. Throughout the diazotization an excess of hydrochloric acid should be present as shown by pH paper. In another beaker a paste is made from N,N-dimethylaniline (0.11 mole) and 200 mL of water. The suspension is cooled to 18° C. by the addition of ice. With vigorous mechanical agitation the diazonium salt solution is run into the N,N-dimethylaniline suspension rather rapidly. To the reaction mixture is carefully added with NaOH (initially) and Na₂CO₃ until pH is between 5 and 7. The reaction mixture is warmed to room temperature, and stirred to completion as indicated by TLC detection. The resulted mixture is filtered to collect the solid that is air-dried and recrystallized from methanol-water to give the pure desired azo dye.

### Example 2

### Preparation of Compound 2

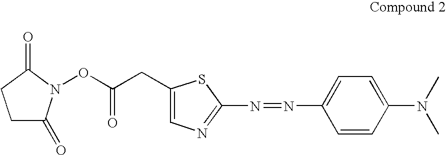

Compound 1 (100 g) is dissolved in N,N-dimethylformamide (DMF, 500 mL). To the solution is added N,N′-disuccinimidyl carbonate (80 g). The solution is stirred while 4-dimethylaminopyridine (500 mg) is added. The reaction mixture is stirred at RT until >90% Compound 1 is consumed (24-36 h, the reaction is followed by TLC every 8 h). The reaction mixture is poured into ice water, and extracted with ethyl acetate. The organic layer is washed with 1% HCl, and dried over Na₂SO₄. The evaporation of ethyl acetate gives the red solid that is washed with ether, and dried under high vacuum to give the desired product.

### Example 3

### Preparation of Compound 3

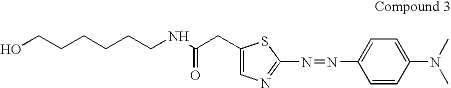

Compound 2 (10 g) is dissolved in acetonitrile (150 mL). To the solution is dropwise and slowly added a solution of 6-aminohexanol (3.5 g) in acetonitrile (50 mL) during the period of 6-8 hours. The mixture is stirred at room temperature overnight. After removal of solvent, the residue is purified on a silica gel column eluted with a gradient of chloroform/ethyl acetate.

### Example 4

### Preparation of Compound 4

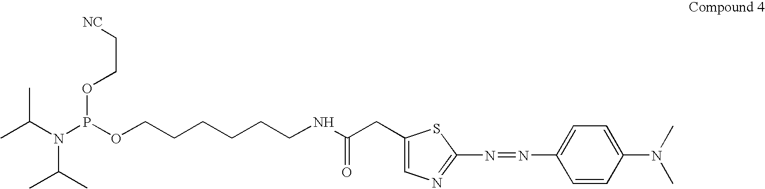

Thoroughly dried Compound 3 (3 g) is dissolved in 100 mL of dry acetonitrile. To the solution 200 mg of tetrazole is added, followed by the phosphitylating agent, bis-(N,N-diisopropyl)-beta-cyanoethyl phosphordiamidite (2.5 g). The reaction is monitored by TLC until the starting material is consumed. The solvent is evaporated and the flask evacuated under high vacuum for two hours. The resulting solid is triturated with dry ether at least five times, until the color of the ether is no longer colorful. The solid is then dried under high vacuum overnight and stored under argon at −20° C.

### Example 5

### Preparation of Compound 5

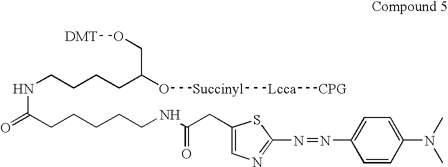

To a solution of N-FMOC-O-DMT-6-amino-1,2-hexanediol (3 g) and 4-dimethylaminopyridine (200 mg) in anhydrous pyridine (15 mL) is added succinic anhydride (300 mg). The reaction is stirred at room temperature overnight. The consumption of starting material is followed by TLC. The mixture is diluted in ethyl acetate (100 mL), washed with 0.5 M sodium chloride (3×100 mL) and saturated sodium chloride (100 mL), and dried over anhydrous sodium sulfate. After concentrating by rotary evaporation and drying under high vacuum, a yellow solid is obtained.

The yellow solid is dissolved in dry dioxane (10 mL) containing anhydrous pyridine (0.5 mL) and p-nitrophenol (350 mg). Dicyclohexylcarbodiimide (1.0 g) is added and the mixture is stirred at room temperature. The reaction is monitored by TLC and after 3 hours, the dicyclohexylurea is collected by filtration. Long chain alkylamine CPG (5.0 g) is suspended in the filtrate containing the p-nitrophenyl ester derivative, triethylamine (1.0 mL) is added, and the mixture is shaken overnight at room temperature. The derivatized support is copiously washed with dimethylformamide, methanol, and diethyl ester and dried in vacuo. Before capping the unreacted alkylamine groups, the loading capacity of the DMT-containing CPG is assayed by determining the amount of dimethoxytrityl cation released upon treatment with perchloric acid according to published procedures (Oligonucleotide Synthesis: A Practical Approach, M. J. Gait (ed.), IRL Press, Oxford, 1984).

Finally, the DMT-containing CPG is achieved by treatment with acetic anhydride-pyridine-DMAP (10:90:1. v/v/w) for one hour. The support is thoroughly washed with methanol and diethyl ether and dried under high vacuum to give the desired DMT-containing CPG. The capped CPG gives a negative ninhydrin test. The FMOC group of the capped CPG is then cleaved as described in the art (U.S. Pat. No. 5,401,837 to P. S. Paul et al.). The FMOC-cleaved LCCA-CPG (5.0 g) is suspended in 10 mL of DMF solution containing Compound 2 (500 mg) and N,N-diisopropylethylamine (1.0 ml), and the mixture is shaken overnight at room temperature. The derivatized support is copiously washed with dimethylformamide, methanol, and diethyl ether and dried in vacuo. Before capping the unreacted alkylamine groups, the loading capacity of the dye-labeled CPG is assayed by determining the amount of dimethoxytrityl cation released upon treatment with perchloric acid according to published procedures (Oligonucleotide Synthesis: A Practical Approach, M. J. Gait (ed.), IRL Press, Oxford, 1984). Finally, capping of the dye-labeled CPG is achieved by treatment with acetic anhydride-pyridine-DMAP (10:90:1. v/v/w) for one hour. The support is thoroughly washed with methanol and diethyl ether and dried under high vacuum to give the dye-labeled CPG that give a negative ninhydrin test.

### Example 6

### Preparation of Compound 6

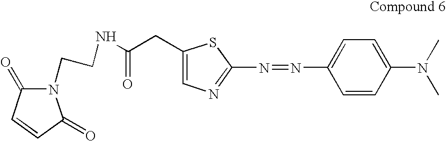

To Compound 2 in acetonitrile at room temperature is added 4 equivalents of triethylamine and 1.2 equivalents of N-(2-aminoethyl)maleimide, trifluoroacetic acid salt (ABD Bioquest). The mixture is stirred at ambient temperature for 15 minutes. The product is precipitated with 4 volumes of ethyl acetate and purified by HPLC.

### Example 7

### Preparation of Compound 7

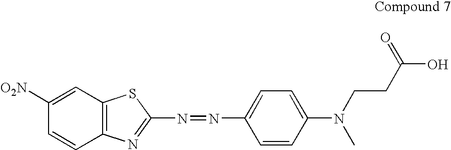

Compound 7 is analogously prepared from the reaction 2-amino-6-nitrobenzothiazole with N-methyl-N-carboxyethylaniline according to the procedure of Compound 1.

### Example 8

### Preparation of Compound 8

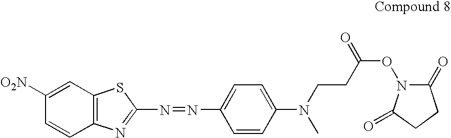

Compound 8 is analogously prepared from Compound 7 according to the procedure of Compound 2.

### Example 9

### Preparation of Compound 9

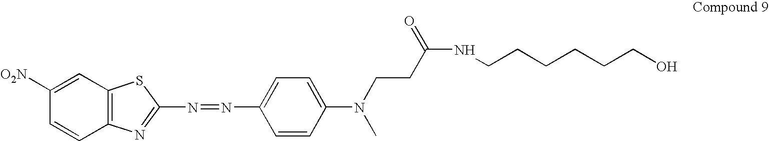

Compound 9 is analogously prepared from the reaction of Compound 8 with 6-aminohexanol according to the procedure of Compound 3.

### Example 10

### Preparation of Compound 10

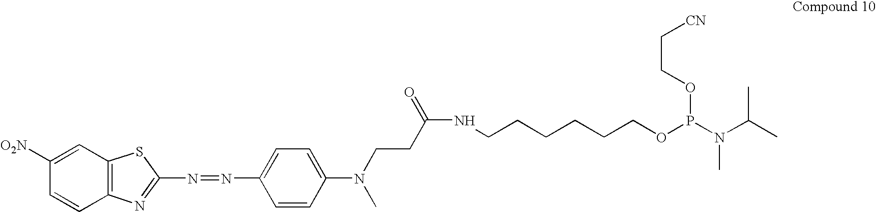

Compound 10 is analogously prepared from Compound 9 according to the procedure of Compound 4.

### Example 11

### Preparation of Compound 11

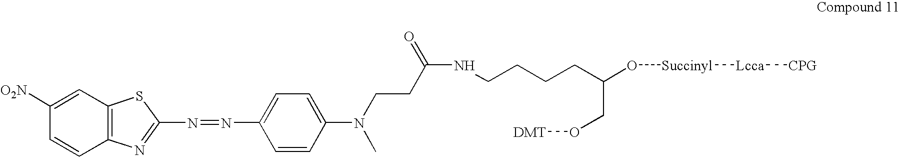

Compound 11 is analogously prepared from Compound 8 according to the procedure of Compound 5.

### Example 12

### Preparation of Compound 12

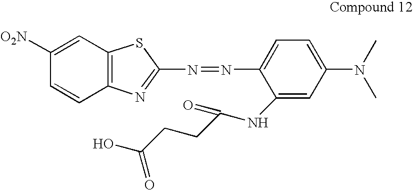

Compound 12 is analogously prepared from the reaction of 2-amino-6-nitrobenzothiazole and 3-(1′-oxo-3′-carboxypropylamino)-N,N-dimethylaniline according to the procedure of Compound 1.

### Example 13

### Preparation of Compound 13

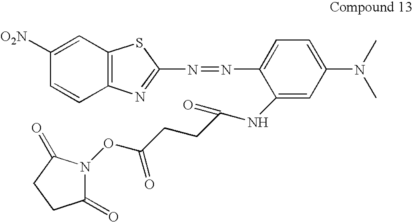

Compound 13 is analogously prepared from Compound 12 according to the procedure of Compound 2.

### Example 14

### Synthesis of an Azo Dye-Labeled FRET Peptide

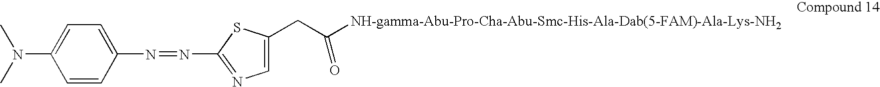

The above FRET peptide is synthesized from Compound 1 by the standard FMOC solid phase synthesis as described in Fmoc Solid Phase Peptide Synthesis: A Practical Approach, by Weng C. Chan, Oxford University Press, 2003. Compounds 7, 12, 22, 23, 24, 33, 34 and 37-42 can be used to replace Compound 1 to label the above peptide sequence with the similar chemistry.

### Example 15

### Synthesis of an Azo Dye-Labeled Oligonucleotide Using Azo Dye Phosphoramidites

Oligonucleotide synthesis is performed using an automated DNA synthesizer according to manufacturer's instructions. Compounds 4, 10, 27 and 31 are used to label oligonucleotides. In each final coupling cycle, the Trityl ON configuration is used. After assembly, the oligonucleotides are cleaved from the support using concentrated ammonia using the manufacturer's end procedure cycle. The residue is dissolved in acetic acid/water (8:2) and the mixture is evaporated to dryness after 20 minutes at room temperature. To the residue is added water (0.5 ml) and the resultant suspension filtered. The aqueous solution now contains the deprotected oligonucleotide ready for purification.

Compound 4, 10, 27 or 31 (100 mg) is dissolved in 1 mL of dry acetonitrile and placed on the DNA synthesizer. Following the procedure suggested by the manufacturer, 50 μL of the solution of Compound 4, 10, 27 or 31 is delivered to the reaction column with 100 μL of a 0.5 M tetrazole activator solution. The mixture is cycled over the support containing the 5′-OH oligonucleotide for a few minutes. Following the removal of excess Compound 4, 10, 27 or 31, the typical coupling cycle is completed by oxidation, capping, and detritylation. Labeled oligonucleotides (5 mer to 10 mer lengths) are released from the solid support and deprotected by treating with concentrated ammonium hydroxide for 20 minutes at 60° C. The azo dye-labeled oligonucleotide are purified and analyzed by TLC (Kieselgel 60 F254 in 55:10:35 isopropanol:water:ammonia) or by reverse phase HPLC (gradient of 10-40% A in B over 30 minutes; A=acetonitrile, B=0.1M triethylammonium acetate, pH 7) or by polyacrylamide gel electrophoresis according to standard procedures (Sambrook et al., Molecular Cloning: A Laboratory Manual, 2nd Edition, Cold Spring Harbor Laboratory Press, 1989).

A derivative such as DMT-Dye-deoxyuridine phosphoramidite (DMT-dye-dU-CEP) may be used to add a dye-labeled deoxyuridine (dU) residue to an oligonucleotide at the 5′ end or any point within the sequence between the 5′ and 3′ ends during the automated oligosynthesis. Usually, a researcher would substitute the dye-dU for a thymidine (dT) in the sequence so that the hybridization base pairing is not affected.

Additionally used derivatives are dye-deoxynucleotide triphosphate (dye-dNTP), dye-ribonucleotide triphosphate (dye-NTP), and dye-dideoxynucleotide triphosphate (dye-ddNTP) compounds. These reagents are useful to label DNA or RNA by enzymatic incorporation of the dye-linked dNTP or NTP. The dye-labeled dideoxynucleotide triphosphates (ddNTP) may be incorporated enzymatically into DNA for DNA sequencing applications as a chain terminator in the Sanger dideoxy sequencing method (Sanger et al., J. Mol. Biol., 143, pp. 161-178, 1980). There is prior art for these compounds. Dye-ddATP, dye-ddCTP, dye-ddGTP, dye-ddTTP analogs are also contemplated.

### Example 16

### Synthesis of an Azo Dye-Labeled Oligonucleotide Using Dye CPG Polymers

Oligonucleotide synthesis is performed using an automated DNA synthesizer according to manufacturer's instructions. Compounds 5, 11, 28 and 32 are used to label oligonucleotides. Azo dye controlled pore glass (CPG) supports (such as Compounds 5, 11, 28 and 32) are used for attachment of quencher dyes to the 3′ terminus of nucleic acids. 5′ fluorophore labeling is accomplished using fluorophore phosphoramidites. Cleavage and deprotection of azo dye oligos is carried out in ammonia at 60° C. with the exception of 3′ TAMRA oligos which is deprotected in 1:3 t-butylamine:water. Following deprotection, dual labeled probes are purified by HPLC. Purified probes are analyzed by both anion exchange and reverse phase HPLC. The dual labeled FRET oligonucleotides are analyzed and used as Melecular Beacons as described in the art (D. P. Bratu (2006), Methods Mol Biol 319, 1-14; S. A. E. Marras (2006), Methods Mol Biol 335, 3-16; S. A. E. Marras, S. Tyagi, and F. R. Kramer (2006), Clin Chim Acta 363, 48-60; A. P. Silverman and E. T. Kool (2005), Trends Biotechnol 23, 225-230).

### Example 17

### Preparation of Oligonucleotide Conjugates of Quenching Compounds

Eighteen-base oligonucleotide conjugates of quencher dyes are prepared using standard methods. Typically, a 6-(N-trifluoroacetylamino)hexyl is synthetically incorporated on the 5′ end of the oligonucleotide of interest as a phosphoramidite, and the TFA protection group is then removed under basic conditions. The resulting conjugate is subsequently reacted with a succinimidyl ester derivative of a quenching compound of the disclosure. Specifically, the succinimidyl ester derivative is dissolved in DMSO at a concentration of about 12.5 mg/mL. The amine-modified oligonucleotide is dissolved in water at a concentration of 25 mg/mL. A fresh solution of 0.1 M sodium borate, pH 8.5 is prepared. In a microfuge tube, 4 μL of the oligonucleotide solution is combined with 200 μg of the quenching compound solution and 100 μL sodium borate buffer. Samples are incubated 4 hours to overnight at room temperature, and the nucleic acids are precipitated by addition of 0.1 volume 0.3 M NaCl and 2.5 volumes cold absolute ethanol. Samples are incubated for 30 minutes at −20° C. and centrifuged in a microfuge for 30 minutes. The supernatant fluid is decanted and the pellet dried under vacuum.

Alternatively, the oligonucleotide conjugate may be prepared by reaction of a maleimide derivative of a quenching compound of the disclosure with an oligonucleotide that has been derivatized by a thiol that has been incorporated via a phosphoramidite.

Conjugates may be purified by reverse phase HPLC, using a C18 reverse phase column and a gradient of 5-95% acetonitrile in 0.1 M TEAA, pH 7. Absorbance and fluorescence emission spectra are determined in 10 mM Tris-HCl, 1 mM EDTA, pH 7.5. Oligonucleotide conjugates of quenching compounds may be weakly fluorescent or substantially nonfluorescent.

### Example 18

### Preparation of an Oligonucleotide Conjugate, Substituted with Both a Fluorophore and a Quenching Compound

Oligonucleotides conjugated to a Cy3 fluorophore at one terminus and a quenching compound of the disclosure at the other terminus are prepared using an azo dye phosphoramidite and an amine modifier at the other terminus of the oligonucleotide, followed by labeling with a succinimidyl ester derivatives of the disclosure, or by synthesis of oligonucleotides containing an amino modifier on one terminus and a thiol at the other terminus, followed by sequential reaction with a maleimide derivative and succinimidyl ester derivative of the fluorophore and quenching compound, or vice versa. The fluorescence of the resulting conjugates is measured at equal conjugate concentration. Selected compounds of the instant disclosure quench the fluorescence of fluorescein much more efficiently than does DABCYL.

### Example 19

### Hybridization of Doubly-Labeled Oligonucleotide Conjugates to Unlabeled Complementary Oligonucleotides

Solutions are prepared containing 1 μg/mL 18-base oligonucleotide conjugates of a quenching compound of the disclosure attached to the 5′ terminus, as well as a Cy3 fluorophore on the 3′ terminus. The oligonucleotide conjugates are hybridized with 40 μg/mL reverse complement oligonucleotide in TE buffer at pH 9.0. The samples are heated for 10 minutes at 65° C., allowed to cool slowly to room temperature, and are then incubated at room temperature for 60 minutes, protected from light. A portion of each sample is transferred to a microplate well and the fluorescence emission of the sample is determined at −570 nm (with 550 nm excitation). The fluorescence is compared to the fluorescence of a buffer solution alone. The conjugates of the disclosure exhibit an increase in fluorescence upon hybridization. Because the quenched oligonucleotides initially exhibited extremely low fluorescence, they show larger increases upon hybridization, and therefore the conjugates that are the most efficiently quenched prior to hybridization typically exhibit the largest increase in fluorescence. This property may be utilized to formulate a homogenous assay method to detect the presence of the specific complementary DNA sequences in a sample. Several of the compounds of the disclosure quench fluorescence more efficiently than DABCYL in this application. Similarly, doubly labeled oligonucleotides that form structures that enhance quenching, such as hairpin or stem loop structures, as in BEACON probes, can also be used in this application.

### Example 20

### Hybridizing Oligonucleotide Conjugates of Quenching Compounds with Fluorophore Labeled Oligonucleotides

Oligonucleotides conjugated to a quenching compound at one terminus quench the fluorescence of fluorophore labeled nucleotides upon hybridization. Labeled oligonucleotides are prepared as described above (Examples 17 and 18), and hybridized with their reverse complements. Samples containing 2 μg/mL quenching compound-labeled 18 base oligonucleotides and 200 ng/mL Cy3-labeled reverse complement oligonucleotides in 10 mM Tris-HCl, 1 mM EDTA, pH 9.0, are hybridized and their fluorescence is determined as described above (Example 19). The quenching compound oligonucleotides efficiently quench the fluorescence of Cy3 that is localized at the same end of hybridized oligonucleotides, but quench the fluorescence of distant fluorophores more poorly.

### Example 21

### Quenching Fluorescence of Nucleotides Added Enzymatically to the 3′ End of a Primer

An eighteen-base oligonucleotide is labeled with Compound 2 on its 5′ terminus, as described in Example 17. The resulting conjugate is incubated with terminal deoxynucleotidyl transferase under standard assay conditions for 3′ end elongation, in the presence of fluorophore-labeled dUTP conjugates, as follows: The oligonucleotide conjugate (650 ng) is incubated with 1 μL of 25 mM fluorophore-labeled nucleotide, 0.5 mM CoCl₂, and 0.2 M potassium cacodylate, 25 mM Tris-HCl, pH 6.6, 2 mM DTT, and 250 μg/mL bovine serum albumin for 60 minutes at 37° C. A one-fifth volume of a solution containing 50% glycerol and 0.01% bromophenol blue is added to each reaction, and the samples are separated by electrophoresis on a 20% polyacrylamide/8 M urea minigel in TBE buffer (45 mM Tris-borate, 1 mM EDTA), under conditions that resolve single nucleotide additions to the oligonucleotide. Samples containing oligonucleotides that are lacking the quenching compound are processed in parallel, for use as size standards. Gels are visualized using a 300-nm UV transilluminator combined with Polaroid black and white photography, or using a laser scanner. The gels are post-stained with a fluorescent nucleic acid stain, such as ethidium bromide, and band fluorescence is visualized in the same way. The size of the oligonucleotides is determined based on comparisons of electrophoretic migration with the unlabeled standard. Quenching is detected as lack of fluorescence or visibility of a band of a particular size from the pattern visible in the standard. Where the fluorophore is Cy3 dye, the label fluorescence is readily quenched by the 5′-bound quenching compound.

This technique is useful as a gel-based method for quantitating terminal transferase activity. Enzyme activity in an unknown sample is determined by comparison of the number of added nucleotides per template or the number of templates with added nucleotides of a certain length with the numbers obtained using a standard amount of enzyme activity following a standard reaction time interval.

### Example 22

### Quenching of a Fluorescent Oligonucleotide by Enzymatic Incorporation of a Quenching Compound Conjugate of Nucleotide Triphosphate Via Primer Extension

A short oligonucleotide, having 6 to about 20 bases, is labeled with a fluorophore such as Cy5 dye, on its 5′ terminus, and then purified via HPLC. For template-driven reactions, the oligonucleotide is hybridized to an appropriate template, and incubated with a quenching compound-labeled nucleotide or deoxynucleotide in an appropriate buffered solution, in the presence of samples thought to contain an appropriate DNA or RNA polymerase. Enzyme activity is determined by measuring the rate of fluorescence loss from the solution, versus the rate of loss observed from solutions containing known amounts of enzyme activity. Terminal deoxynucleotidyltransferase activity is assayed by determining the rate of fluorescence loss from the solution upon incubation with samples thought to contain terminal deoxynucleotidyltransferase activity. For measurement of terminal deoxynucleotidyl transferase activity, fluorophore-labeled templates are incubated with quenching compound-labeled nucleotides for a set time interval, and fluorescence is measured in a fluorescence microplate reader or fluorometer.

To measure reverse transcriptase activity, 2 μg mRNA is combined with 5 μg fluorophore labeled poly dT(16) oligomer in 10 mM Tris-HCl, pH. 8.0, 1 mM EDTA; the mixture is heated to 70° C. for 10 minutes and then chilled on ice. A solution containing 2 μL reverse transcriptase (200 units/μL for the standard, or unknown amounts), 500 uM dATP, 500 uM dCTP, 500 uM dGTP, 200 uM dTTP, and 60 uM quenching compound-labeled dUTP is prepared and added to the RNA. The reaction is allowed to proceed for 2 hours at 42° C. The fluorescence of the solution is measured in a fluorescence microplate reader or fluorometer versus a standard. The decrease in fluorescence in comparison to samples lacking enzyme activity is directly related to the activity of the enzyme in the reaction.

To measure Klenow DNA polymerase activity, 1 μg random sequence 9-mer oligonucleotides labeled with a fluorescent dye are combined with 2.5 μg genomic DNA in 10 mM Tris-HCl, pH 8.0, 1 mM EDTA. The mixture is boiled for 2 minutes and chilled on ice. A reaction mixture containing 25 uM dATP, 25 uM dCTP, 25 uM dGTP and 10 uM dTTP, plus 40 uM quenching compound-labeled dUTP in 1 mM Tris-HCl, pH 7.5, 5 mM NaCl, 0.01 mM EDTA, pH 8.0, 5 mM dithiothreitol is combined with samples thought to contain DNA polymerase. The reaction mixture is combined with the DNA mixture and incubated at 37° C. for 2 hours.

### Example 23

### Using Quenching Compounds to Measure Nuclease Activity

Oligonucleotide conjugates labeled with both a quenching compound at one terminus and a fluorophore at the other terminus are prepared as described in Examples 17 and 18. For measuring single-stranded nuclease activity, the conjugates are incubated in the presence of samples thought to contain nuclease activity in the presence of an appropriate buffer and the resulting fluorescence increase in the sample is compared to that obtained using standards of known nuclease concentration. To measure double-stranded nuclease activity, double-stranded templates are prepared by hybridizing two oligonucleotides to one another, or by chemically modifying a double-stranded template using reagents such as platinum complexes of fluorophores and quenchers (as described in U.S. Pat. Nos. 5,714,327 and 6,133,038, incorporated by reference), or by using an enzyme such as a terminal transferase to add nucleotides to the end of a template as described in Examples 20 and 21. Samples thought to contain nuclease activity are incubated with such templates in the presence of appropriate buffers and the increase in fluorescence compared to a standard, as described in Example 20.

### Example 24

### Using Quenching Compounds to Measure Ligase Activity

Oligonucleotide hexamers labeled at the 5′ terminus with a quenching compound are prepared as described in Example 17 and 18. Oligonucleotide hexamers labeled with a fluorophore at the 3′ terminus and phosphate at the 5′ terminus are analogously prepared except that the phosphate is alternatively applied by standard methods using a phosphoramidite or by enzymatic means, such as T4 polynucleotide kinase.

A reaction mixture is prepared that contains about 5 μg of each oligonucleotide conjugate, 0.5 mM ATP, and samples thought to contain ligase activity, in 1 mM MgCl₂, 2 mM dithiothreitol, 5 μg/mL bovine serum albumin, and 5 mM Tris-HCl, pH 7.7, in a volume of 20 μL. The reaction mixtures are incubated for 2 hours to overnight at 22° C., and the sample fluorescence is measured. As the quenching compound-labeled oligonucleotides do not contain a free 5′ phosphate, they cannot ligate to one another, and as the fluorophore-labeled oligonucleotides do not contain a free 3′ hydroxyl, they cannot ligate to one another. Thus the only products of ligation will be a dimer of the two oligonucleotides and the fluorescence decrease observed during the course of the reaction is a measure of ligase activity. Alternatively, RNA oligonucleotides are used as templates to measure RNA ligase activity or splicing activity.

### Example 25

### Preparation of Quenched Double-Stranded DNA

Oligonucleotides are prepared that are either labeled with a fluorophore at a strand terminus, or within the oligonucleotide sequence itself, using standard methods as described above. The oligonucleotides are then used as primers for PCR or are otherwise enzymatically extended using standard methods. A quenching compound platinum complex is prepared (as described in U.S. Pat. No. 5,714,327) and dissolved in water at a final concentration of 1 mg/mL. DNA (500 ng) is combined with 1.5 μg of the quenching compound platinum complex and incubated in a total volume of 25 μL water for 15 minutes at 65° C. The reaction is chilled in an ice bath to stop it. The quenched DNA is not visible after gel electrophoresis, even when stained with a fluorescent nucleic acid stain or incubation in solution with a fluorescent nucleic acid stain.

### Example 26

### Using Quenching Compounds to Assay Topoisomerase Activity

Quenched DNA is prepared as described above, using a circular single stranded DNA template, such as an M13 or ØX174 phage DNA genome, and a quenching compound platinum complex (prepared as described in U.S. Pat. No. 5,714,327). A fluorophore-labeled oligonucleotide is then hybridized to the quenched DNA. Samples thought to contain topoisomerase activity are combined with the template under optimal reaction conditions for the enzyme, and the reaction is allowed to proceed for an appropriate period of time. Enzyme activity is measured as fluorescence increase for the solution, using a fluorescence microplate reader or fluorometer.

### Example 27

### Detection of Matrix Metalloproteinase Activities

The matrix metalloproteinases (MMPs) constitute a family of zinc-dependent endopeptidases that function within the extracellular matrix. These enzymes are responsible for the breakdown of connective tissues and are important in bone remodeling, the menstrual cycle and repair of tissue damage. While the exact contribution of MMPs to certain pathological processes is difficult to assess, MMPs appear to have a key role in the development of arthritis as well as in the invasion and metastasis of cancer. 50 μM the FRET peptide is incubated with 4 nM MMPs or without MMPs (control) at room temperature. The fluorescence signal is recorded on a fluorescence microplate reader at Ex/Em=490±30 nm/520±30 nm. The recording is started as soon as the enzymatic reaction is initiated. The result is shown in FIG. 4.

### Example 28

### Detection of HIV Protease Activities Using FRET Peptides

Inhibition of HIV-1 protease represents an important avenue for AIDS therapy. Currently combination chemotherapy of reverse transcriptase inhibitors and protease inhibitors have shown to suppress the replication of HIV-1 and extend the life expectancy of HIV-1-infected individuals. The following FRET peptides of the instant disclosure are screened for developing a fluorogenic HIV protease assay. The enzyme assays are done with purified enzymes. Absorption spectra were taken with Hitachi U-3010. Endpoint fluorescence assays are run on either BMG NovoStar or Gemini SpectraMax (Molecular Devices). Enzyme kinetic assays are run on FlexStation (Molecular Devices). As shown in FIG. 3. FRET peptide #2 demonstrates excellent response to HIV-1 protease. FRET Peptides #6 and #7 might be used for monitoring HIV protease activity in combination with a flow cytometer.

| # | |
|---|---|
| Peptide | Peptide Sequence |
| 1 | Compound 12-Gaba-Ser-Gln-Asn-Tyr-Pro- |
| | Ile-Val-Gln-(5-TAMRA) |
| 2 | Arg-Glu(5-TAMRA)-Val-Ser-Phe-Asn-Phe- |
| | Pro-Gln-Ile-Thr-Lys(Compound 12)-Arg |
| 3 | Arg-Glu(5-TAMRA)-Ser-Gln-Asn-Tyr-Ile- |
| | Val-Gln-Lys(Compound 12)-Arg |
| 4 | Arg-Arg-Glu(5-TAMRA)-Ser-Gln-Asn-Tyr- |
| | Pro-Ile-Val-Gln-Lys(Compound 12)-Arg-Arg |
| 5 | Ac-Arg-Glu(5-TAMRA)-Ser-Gln-Asn-Tyr-Pr- |
| | Ile-Val-Gln-Lys(Compound 12)-Arg-NH2 |
| 6 | (Rhodamine Latex Bead)-[Cys-Val-Ser-Phe- |
| | Asn-Phe-Pro-Gln-Ile-Thr-Lys(Compound 12)- |
| | Arg]n (n > 3) |
| 7 | (Compound 12 Latex Bead)-[Cys-Val-Ser- |
| | Phe-Asn-Phe-Pro-Gln-Ile-Thr-Lys(5-TAMRA)- |
| | Arg]n (n > 3) |

All the above FRET peptides are synthesized using the standard FMOC chemistry. Some of the TQ peptides were prepared using FMOC-Lys (Compound 12)-OH. For the post-labeling of the premade peptides, Dye NHS esters were used for labeling N-terminal amino or ε-amino group of lysine residue. Dye maleimides were used for labeling the SH group of cysteine residues.

### Example 29

### Detection of Intracellular Calcium Using Water-Soluble Azo Dyes

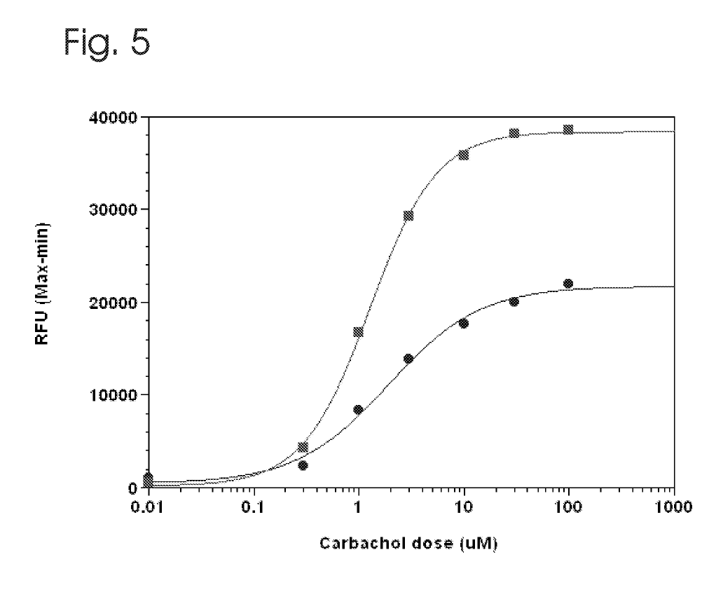

Screen Quest™ Fluo-8 NW Calcium Assay Kit is used in combination with 1 mM Compound 42. The results are shown in FIG. 5. Specifically, the following steps are followed to run the calcium assay:
1) HEK-293 cells are seeded overnight at 40,000 cells per 100 μL per well in a 96-well black wall/clear bottom costar plate.
2) 100 μl DMSO is added into 1 mg Fluo-8 NW to make Fluo-8 NW stock solution.
3) 9 ml HHBS is added into 10× Pluronic F127 Plus (1 mL) to make 1× assay buffer.
4) 10 μl of DMSO-reconstituted Fluo-8 NW is added into 10 mL of 1× assay buffer to make Fluo-8 NW dye-loading solution for one cell plate by adding.
5) Compound 42 (0.1 M aqueous solution) is added into the Fluo-8 NW dye loading solution to 3 mM final concentration in the assay plate well.
6) 100 μL/well (96-well plate) or 25 μL/well (384-well plate) Fluo-8 NW dye-loading solution containing Compound 42 is added to the assay plate.
7) The dye-loading plate is incubated in a cell incubator for 30 minutes, and then incubated at room temperature for another 30 minutes.
8) The compound plates are prepared using HHBS.
9) The calcium flux assay is run by monitoring the fluorescence at Ex=490/Em=525 nm using BMG NOVO Star Microplate Reader.

Although the present invention has been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances that fall within the scope of the appended claims.

What is claimed is:

1. A compound having the formula 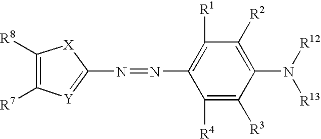
wherein X is O, S, Se, NH, or N—R¹⁰, C—R¹⁰R¹¹;
Y is CH, CH—R⁶, N, or ⁺N—R¹¹;
R¹ to R⁸ are independently hydrogen, alkyl, alkoxy, halogen, alkylthio, cyano, sulfonyl, phosphonyl, boronyl, carbonyl, hydroxy, amino, thiol, aryl, heteroaryl or an RM where each RM is independently a succinimidyl ester, an activated phenol ester, a maleimide, an iodoacetamide, or a phosphoramidite;
R¹⁰ to R¹³ are independently alkyl, polyethylene glycol, aryl or an RM;
or one or more of R¹ and R², R³ and R⁴, and R⁷ and R⁸, or R⁸ and R¹⁰ taken in combination form an aryl or heteroaryl ring;
or one or more of R² and R¹², or R³ and R¹³ taken in combination form a 5- to 8-membered ring;
or R¹² and R¹³ when taken in combination may form a 3- to 12-membered ring;
provided that at least one of R¹ to R¹³ is an RM.

2. A compound according to claim 1 having the formula 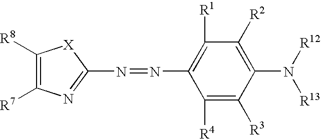
wherein X is O, S, Se, NH, or N—R¹⁰, C—R¹⁰R¹¹;
R¹ to R⁸ are independently hydrogen, alkyl, alkoxy, halogen, alkylthio, cyano, sulfonyl, phosphonyl, boronyl, carbonyl, hydroxy, amino, thiol, aryl, heteroaryl or an RM where each RM is independently a succinimidyl ester, an activated phenol ester, a maleimide, an iodoacetamide, or a phosphoramidite;
R¹⁰ to R¹³ are independently alkyl, polyethylene glycol, aryl or an RM;
or one or more of R¹ and R², R³ and R⁴, and R⁷ and R⁸, or R⁸ and R¹⁰ taken in combination form an aryl or heteroaryl ring;
or one or more of R² and R¹², or R³ and R¹³ taken in combination form a 5- to 8-membered ring;
or R¹² and R¹³ when taken in combination may form a 3- to 12-membered ring;
provided that at least one of R¹ to R¹³ is an RM.

3. A compound according to claim 1 having the formula 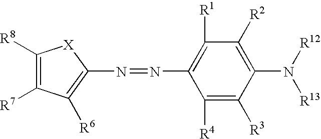
wherein X is O, S, Se, NH, or N—R¹⁰, C—R¹⁰R¹¹;
R¹ to R⁸ are independently hydrogen, alkyl, alkoxy, halogen, alkylthio, cyano, sulfonyl, phosphonyl, boronyl, carbonyl, hydroxy, amino, thiol, aryl, heteroaryl, or an RM where each RM is independently a succinimidyl ester, an activated phenol ester, a maleimide, an iodoacetamide, or a phosphoramidite;
R¹⁰ to R¹³ are independently alkyl, polyethylene glycol, aryl or an RM;
or one or more of R¹ and R², R³ and R⁴, R⁶ and R⁷, R⁷ and R⁸, or R⁸ and R¹⁰ when taken in combination form an aryl or heteroaryl ring;
or R² and R¹², or R³ and R¹³ when taken in combination form a 5- to 8-membered ring;
or R¹² and R¹³ when taken in combination form a 3- to 12-membered ring;
provided that at least one of R¹ to R¹³ is an RM.

4. A compound according to claim 3, wherein at least one of R⁶, R⁷, and R⁸ is a thiophene, a bithiophene, a terthiophene, a furan, an imidazole, an oxazole, a thiazole, a pyrazole, an imidazole, a triazole or a terazole, a pyrole, or an indoline.

5. A compound according to claim 1 having the formula 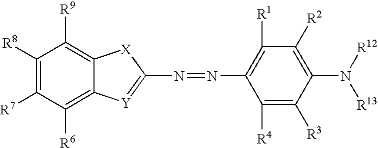
wherein X is O, S, Se, NH, or N—R¹⁰, C—R¹⁰R¹¹;
R¹ to R⁹ are independently hydrogen, alkyl, alkoxy, halogen, alkylthio, cyano, sulfonyl, phosphonyl, boronyl, carbonyl, hydroxy, amino, thiol, aryl, heteroaryl, or an RM where each RM is RM is independently a succinimidyl ester, an activated phenol ester, a maleimide, an iodoacetamide, or a phosphoramidite;
R¹⁰ to R¹³ are independently alkyl, polyethylene glycol, aryl or an RM;
or one or more of R¹ and R², R³ and R⁴, R⁶ and R⁷, R⁷ and R⁸, R⁸ and R⁹, or R⁹ and R¹⁰ when taken in combination form an aryl or heteroaryl ring;
or one or more of R² and R¹², or R³ and R¹³ when taken in combination form a 5- to 8-membered ring;
or R¹² and R¹³ when taken in combination form a 3- to 12-membered ring;
provided that at least one of R¹ to R¹³ is an RM.

6. A compound according to claim 1 having the formula 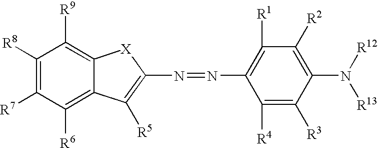
wherein X is O, S, Se, NH, or N—R¹⁰, C—R¹⁰R¹¹;
R¹ to R⁹ are independently hydrogen, alkyl, alkoxy, halogen, alkylthio, cyano, sulfonyl, phosphonyl, boronyl, carbonyl, hydroxy, amino, thiol, aryl, heteroaryl, or an RM where each RM is independently a succinimidyl ester, an activated phenol ester, a maleimide, an iodoacetamide, or a phosphoramidite;
R¹⁰ to R¹³ are alkyl, polyethylene glycol, aryl or an RM;
or one or more of R¹ and R², R³ and R⁴, R⁵ and R⁶, R⁶ and R⁷, R⁷ and R⁸, R⁸ and R⁹, or R⁹ and R¹⁰ when taken in combination form an aryl or heteroaryl ring;
or one or more of R² and R¹² or R³ and R¹³ taken in combination form a 5- to 6-membered ring;
or R¹² and R¹³ when taken in combination form a 3- to 12-membered ring;
provided that at least one of R¹ to R¹³ is an RM.

7. A compound according to claim 1 having the formula 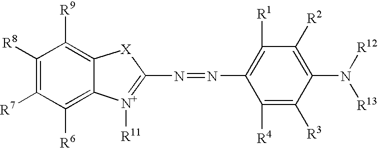
wherein X is O, S, Se, NH, or N—R¹⁰, C—R¹⁰R¹¹;
R¹ to R⁹ are independently hydrogen, alkyl, alkoxy, halogen, alkylthio, cyano, sulfonyl, phosphonyl, boronyl, carbonyl, hydroxy, amino, thiol, aryl, heteroaryl, or an RM where each RM is independently a succinimidyl ester, a maleimide or a phosphoramidite;
R¹⁰ to R¹³ are independently alkyl, polyethylene glycol, aryl, or an RM;
or one or more of R¹ and R², R³ and R⁴, R⁶ and R⁷, R⁷ and R⁸, R⁸ and R⁹, R⁶ and R¹¹, or R⁹ and R¹⁰ when taken in combination form an aryl or heteroaryl ring;
or one or more of R² and R¹², and R³ and R¹³, or R⁶ and R¹¹ taken in combination form a 5- to 8-membered ring;
or R¹² in combination with R¹³ forms a 3- to 12-membered ring;
provided that at least one of R¹ to R¹³ is an RM.

8. A compound having the formula 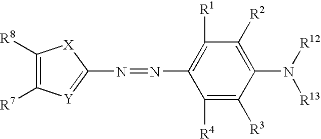
wherein X is O, S, Se, NH, or N—R¹⁰, C—R¹⁰R¹¹;
Y is CH, CH—R⁶, N, or ⁺N—R¹¹;
R¹ to R⁸ are independently hydrogen, alkyl, alkoxy, halogen, alkylthio, cyano, sulfonyl, phosphonyl, boronyl, carbonyl, hydroxy, amino, thiol, aryl, heteroaryl or a SUBSTRATE where each SUBSTRATE is a polymer used for synthesizing one of peptides and oligonucleotides;
R¹⁰ to R¹³ are independently alkyl, polyethylene glycol, aryl or a SUBSTRATE;
or one or more of R¹ and R², R³ and R⁴, and R⁷ and R⁸, or R⁸ and R¹⁰ taken in combination form an aryl or heteroaryl ring;
or one or more of R² and R¹², or R³ and R¹³ taken in combination form a 5- to 8-membered ring;
or R¹² and R¹³ when taken in combination may form a 3- to 12-membered ring;
provided that at least one of R¹ to R¹³ is an SUBSTRATE.

9. A compound according to claim 8 having the formula 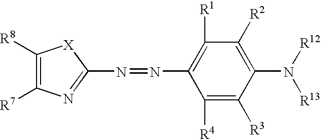
wherein X is O, S, Se, NH, or N—R¹⁰, C—R¹⁰R¹¹;
R¹ to R⁸ are independently hydrogen, alkyl, alkoxy, halogen, alkylthio, cyano, sulfonyl, phosphonyl, boronyl, carbonyl, hydroxy, amino, thiol, aryl, heteroaryl, or a SUBSTRATE where SUBSTRATE is a polymer used for synthesizing one of peptides and oligonucleotides;
R¹⁰ to R¹³ are independently alkyl, polyethylene glycol, aryl or a SUBSTRATE;
or one or more of R¹ and R², R³ and R⁴, and R⁷ and R⁸, or R⁸ and R¹⁰ taken in combination form an aryl or heteroaryl ring;
or one or more of R² and R¹², or R³ and R¹³ taken in combination form a 5- to 8-membered ring;
or R¹² and R¹³ when taken in combination may form a 3- to 12-membered ring;
provided that at least one of R¹ to R¹³ is a SUBSTRATE.

10. A compound according to claim 9, wherein at least one SUBSTRATE is a Controlled Pore Glass (CPG) support useful for synthesizing oligonucleotides.

11. A compound according to claim 8 having the formula 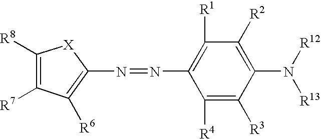
wherein X is O, S, Se, NH, or N—R¹⁰, C—R¹⁰R¹¹;
R¹ to R⁸ are independently hydrogen, alkyl, alkoxy, halogen, alkylthio, cyano, sulfonyl, phosphonyl, boronyl, carbonyl, hydroxy, amino, thiol, aryl, heteroaryl, or a SUBSTRATE where each SUBSTRATE is a polymer useful for synthesizing peptides or oligonucleotides;
R¹⁰ to R¹³ are independently alkyl, polyethylene glycol, aryl or a SUBSTRATE;
or one or more of R¹ and R², R³ and R⁴, R⁶ and R⁷, R⁷ and R⁸, or R⁸ and R¹⁰ when taken in combination form an aryl or heteroaryl ring;
or R² and R¹², or R³ and R¹³ when taken in combination form a 5- to 8-membered ring;
or R¹² and R¹³ when taken in combination form a 3- to 12-membered ring;
provided that at least one of R¹ to R¹³ is a SUBSTRATE.

12. A compound according to claim 11, wherein at least one SUBSTRATE is a Controlled Pore Glass (CPG) support useful for synthesizing oligonucleotides.

13. A compound according to claim 8 having the formula 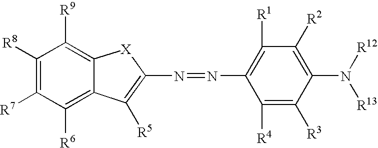
wherein X is O, S, Se, NH, or N—R¹⁰, C—R¹⁰R¹¹;
R¹ to R⁹ are independently hydrogen, alkyl, alkoxy, halogen, alkylthio, cyano, sulfonyl, phosphonyl, boronyl, carbonyl, hydroxy, amino, thiol, aryl, heteroaryl, or a SUBSTRATE where each SUBSTRATE is independently a polymer useful for synthesizing peptides or oligonucleotides;
R¹⁰ to R¹³ are independently alkyl, polyethylene glycol, aryl or a SUBSTRATE;
or one or more of R¹ and R², R³ and R⁴, R⁵ and R⁶, R⁶ and R⁷, R⁷ and R⁸, R⁸ and R⁹, or R⁹ and R¹⁰ when taken in combination form an aryl or heteroaryl ring;
or one or more of R² and R¹², or R³ and R¹³ taken in combination form a 5- to 6-membered ring;
or R¹² and R¹³ when taken in combination form a 3- to 12-membered ring;
provided that at least one of R¹ to R¹³ is a SUBSTRATE.

14. A compound according to claim 13, wherein at least one SUBSTRATE is a Controlled Pore Glass (CPG) support useful for synthesizing oligonucleotides.

15. A compound according to claim 8 having the formula 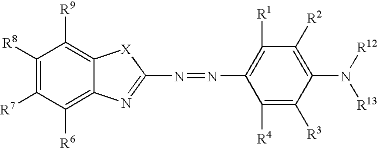
wherein X is O, S, Se, NH, or N—R¹⁰, C—R¹⁰R¹¹;
R¹ to R⁹ are independently hydrogen, alkyl, alkoxy, halogen, alkylthio, cyano, sulfonyl, phosphonyl, boronyl, carbonyl, hydroxy, amino, thiol, aryl, heteroaryl, or a SUBSTRATE where each SUBSTRATE is independently a polymer useful for synthesizing peptides or oligonucleotides;
R¹⁰ to R¹³ are independently alkyl, polyethylene glycol, aryl, or a SUBSTRATE;
or one or more of R¹ and R², R³ and R⁴, R⁶ and R⁷, R⁷ and R⁸, R⁸ and R⁹, or R⁹ and R¹⁰ when taken in combination form an aryl or heteroaryl ring;
or one or more of R² and R¹², or R³ and R¹³ when taken in combination form a 5- to 8-membered ring;
or R¹² and R¹³ when taken in combination form a 3- to 12-membered ring;
provided that at least one of R¹ to R¹³ is a SUBSTRATE.

16. A compound according to claim 15, wherein at least one SUBSTRATE is a Controlled Pore Glass (CPG) support useful for synthesizing oligonucleotides.

17. A compound according to claim 8 having the formula 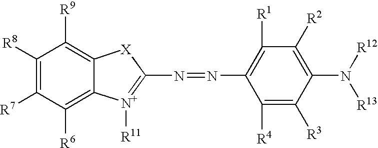
wherein X is O, S, Se, NH, or N—R¹⁰, C—R¹⁰R¹¹;
R¹ to R⁹ are independently hydrogen, alkyl, alkoxy, halogen, alkylthio, cyano, sulfonyl, phosphonyl, boronyl, carbonyl, hydroxy, amino, thiol, aryl, heteroaryl or a SUBSTRATE where each SUBSTRATE is independently a polymer useful for synthesizing peptides or oligonucleotides;
R¹⁰ to R¹³ are independently alkyl, polyethylene glycol, aryl or a SUBSTRATE;
or one or more of R¹ and R², R³ and R⁴, R⁶ and R⁷, R⁷ and R⁸, R⁸ and R⁹, R⁶ and R¹¹, or R⁹ and R¹⁰ when taken in combination form an aryl or heteroaryl ring;
or one or more of R² and R¹², and R³ and R¹³, or R⁶ and R¹¹ taken in combination form a 5- to 8-membered ring;
or R¹² in combination with R¹³ forms a 3- to 12-membered ring;
provided that at least one of R¹ to R¹³ is a SUBSTRATE.

18. A dye-conjugate having the formula 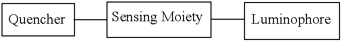
wherein Quencher is a quenching compound according to any one of claims 1-7;
Sensing Moiety is a substance capable of binding to, or being cleaved by an analyte of interest; and
Luminophore is a luminescent dye;
where the Sensing Moiety is bound to both the Quencher and the Luminophore.

19. A dye-conjugate according to claim 18, wherein the Sensing Moiety includes a peptide, a nucleotide, a protein, a nucleic acid or a carbohydrate.

20. A dye-conjugate according to claim 18, wherein the Luminophore is a fluorescein, a coumarin, a rhodamine, a cyanine, an oxazine, a bodipy, a naphthalene, a lanthanide complex, or a ruthenium complex.

21. A dye-conjugate according to claim 18, wherein the analyte of interest is a protease enzyme.

22. A dye-conjugate according to claim 18, wherein the analyte of interest is a nucleotide capable of binding to the Sensing Moiety.

23. A method of detecting an analyte, comprising:
a). preparing a sample that contains a dye-conjugate having the formula 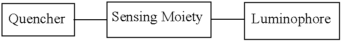
wherein the Quencher is derived from a compound according to any one of claims 1-7;
the Luminophore is a luminescent dye;
the Sensing Moiety is a substance capable of responding to a preselected environmental condition by changing a separation distance between the Quencher and the Luminophore; and
the Sensing Moiety is bound to both the Quencher and the Luminophore;
b). detecting a first luminescence response of said sample;
c) exposing the sample to an experimental environmental condition that is sufficient to change, or thought to be sufficient to change, the separation distance between the quencher and the Luminophore;
d). detecting a second luminescence response of said sample;
e). determining a difference between said first and second luminescence responses;
f). correlating said difference to a change in the separation distance between said Quencher and said Luminophore;
and
g). correlating said change in the separation distance between said Quencher and said Luminophore with said experimental environmental condition.

24. An assay kit, comprising at least:
a). a dye-conjugate according to claim 18; and
b). a biological buffer.
